(19) United States
(12) Patent Application Publication
(10) Pub. No.: US 2002/0165157 A1
(43) Pub. Date: Nov. 7, 2002

(54) Apo-2LI and Apo-3 polypeptides

(75) Inventor: Avi J. Ashkenazi, San Mateo, CA (US)

Correspondence Address: GENENTECH, INC., 1 DNA WAY, SOUTH SAN FRANCISCO, CA, 94080, US

(73) Assignee: Genentech, Inc.

(21) Appl. No.: 10/081,280

(22) Filed: Feb. 21, 2002

Related U.S. Application Data
(63) Continuation of application No. 08/829,270, filed on Mar. 31, 1997, now abandoned.
(60) Provisional application No. 60/014,699, filed on Apr. 1, 1996.
(60) Provisional application No. 60/026,943, filed on Sep. 23, 1996.

(51) Int. Cl. A61K 38/17; C07K 14/775; C07K 16/18

(52) U.S. Cl. 514/12; 530/359; 530/388.25

(57) ABSTRACT

Novel polypeptides, designated Apo-3 and Apo-2LI, involved in apoptosis are provided. Compositions including chimeric molecules, nucleic acids, and antibodies are also provided.

### RELATED APPLICATIONS

This is a non-provisional application claiming priority under 35 USC 119(e) to provisional application Nos. 60/014,699 filed Apr. 1, 1996 and 60/026,943 filed Sep. 23, 1996, the contents of which are incorporated herein by reference.

### FIELD OF THE INVENTION

The present invention relates generally to the identification, isolation, and recombinant production of novel polypeptides involved in mammalian cell apoptosis. In particular, polypeptides designated herein as “Apo-3” and certain forms thereof designated herein as “Apo-2LI” are disclosed. Methods of employing the polypeptides of the invention are also disclosed.

### BACKGROUND OF THE INVENTION

Control of cell numbers in mammals is believed to be determined, in part, by a balance between cell proliferation and cell death. One form of cell death, sometimes referred to as necrotic cell death, is typically characterized as a pathologic form of cell death resulting from some trauma or cellular injury. In contrast, there is another, “physiologic”form of cell death which usually proceeds in an orderly or controlled manner. This orderly or controlled form of cell death is often referred to as “apoptosis” [see, e.g., Barr et al., Bio/Technology, 12:487-493 (1994)]. Apoptotic cell death naturally occurs in many physiological processes, including embryonic development and clonal selection in the immune system [Itoh et al., Cell, 66:233-243 (1991)]. Decreased levels of apoptotic cell death have been associated with a variety of pathological conditions, including cancer, lupus, and herpes virus infection [Thompson, Science, 267:1456-1462 (1995)]. Increased levels of apoptotic cell death may be associated with a variety of other pathological conditions, including AIDS, Alzheimer's disease, Parkinson's disease, amyotrophic lateral sclerosis, multiple sclerosis, retinitis pigmentosa, cerebellar degeneration, aplastic anemia, myocardial infarction, stroke, reperfusion injury, and toxin-induced liver disease [see, Thompson, supra].

Apoptotic cell death is typically accompanied by one or more characteristic morphological and biochemical changes in cells, such as condensation of cytoplasm, loss of plasma membrane microvilli, segmentation of the nucleus, degradation of chromosomal DNA or loss of mitochondrial function. A variety of extrinsic and intrinsic signals are believed to trigger or induce such morphological and biochemical cellular changes [Raff, Nature, 356:397-400 (1992); Steller, Science, 267:1445-1449 (1995); Sachs et al., Blood, 82:15 (1993)]. For instance, they can be triggered by hormonal stimuli, such as glucocorticoid hormones for immature thymocytes, as well as withdrawal of certain growth factors [Watanabe-Fukunaga et al., Nature, 356:314-317 (1992)]. Also, some identified oncogenes such as myc, rel, and ElA, and tumor suppressors, like p53, have been reported to have a role in inducing apoptosis. Certain chemotherapy drugs and some forms of radiation have likewise been observed to have apoptosis-inducing activity [Thompson, supra].

Various molecules, such as tumor necrosis factor-α (“TNF-α”), tumor necrosis factor-β (“TNF-β” or “lymphotoxin”), CD30 ligand, CD27 ligand, CD40 ligand, OX-40 ligand, 4-1BB ligand, Apo-1 ligand (also referred to as Fas ligand or CD95 ligand), TRAIL, and Apo-2 ligand have been identified as members of the tumor necrosis factor (“TNF”) family of cytokines [See, e.g., Gruss and Dower, Blood, 85:3378-3404 (1995); Wiley et al., Immunity, 3:673-682 (1995); Pitti et al., J. Biol. Chem., 271:12687-12690 (1996)]. Among these molecules, TNF-α, TNF-β, CD30 ligand, 4-1BB ligand, Apo-1 ligand, TRAIL, and Apo-2 ligand have been reported to be involved in apoptotic cell death. Both TNF-α and TNF-β have been reported to induce apoptotic death in susceptible tumor cells [Schmid et al., Proc. Natl. Acad. Sci., 83:1881 (1986); Dealtry et al., Eur. J. Immunol., 17:689 (1987)]. Zheng et al. have reported that TNF-α is involved in post-stimulation apoptosis of CD8-positive T cells [Zheng et al., Nature, 377:348-351 (1995)]. Other investigators have reported that CD30 ligand may be involved in deletion of self-reactive T cells in the thymus [Amakawa et al., Cold Spring Harbor Laboratory Symposium on Programmed Cell Death, Abstr. No. 10, (1995)].

Mutations in the mouse Fas/Apo-1 receptor or ligand genes (called lpr and gld, respectively) have been associated with some autoimmune disorders, indicating that Apo-1 ligand may play a role in regulating the clonal deletion of self-reactive lymphocytes in the periphery [Krammer et al., Curr. Op. Immunol., 6:279-289 (1994); Nagata et al., Science, 267:1449-1456 (1995)]. Apo-1 ligand is also reported to induce post-stimulation apoptosis in CD4-positive T lymphocytes and in B lymphocytes, and may be involved in the elimination of activated lymphocytes when their function is no longer needed [Krammer et al., supra; Nagata et al., supra]. Agonist mouse monoclonal antibodies specifically binding to the Apo-1 receptor have been reported to exhibit cell killing activity that is comparable to or similar to that of TNF-α [Yonehara et al., J. Exp. Med., 169:1747-1756 (1989)].

Induction of various cellular responses mediated by such TNF family cytokines is believed to be initiated by their binding to specific cell receptors. Two distinct TNF receptors of approximately 55-kDa (TNFR1) and 75-kDa (TNFR2) have been identified [Hohman et al., J. Biol. Chem., 264:14927-14934 (1989); Brockhaus et al., Proc. Natl. Acad. Sci., 87:3127-3131 (1990); EP 417,563, published Mar. 20, 1991] and human and mouse cDNAs corresponding to both receptor types have been isolated and characterized [Loetscher et al., Cell, 61:351 (1990); Schall et al., Cell, 61:361 (1990); Smith et al., Science, 248:1019-1023 (1990); Lewis et al., Proc. Natl. Acad. Sci., 88:2830-2834 (1991); Goodwin et al., Mol. Cell. Biol., 11:3020-3026 (1991)]. Extensive polymorphisms have been associated with both TNF receptor genes [see, e.g., Takao et al., Immunogenetics, 37:199-203 (1993)]. Both TNFRs share the typical structure of cell surface receptors including extracellular, transmembrane and intracellular regions. The extracellular portions of both receptors are found naturally also as soluble TNF-binding proteins [Nophar, Y. et al., EMBO J., 9:3269 (1990); and Kohno, T. et al., Proc. Natl. Acad. Sci. U.S.A., 87:8331 (1990)]. More recently, the cloning of recombinant soluble TNF receptors was reported by Hale et al. [J. Cell. Biochem. Supplement 15F, 1991, p. 113 (P424)].

The extracellular portion of type 1 and type 2 TNFRs (TNFR1 and TNFR2) contains a repetitive amino acid sequence pattern of four cysteine-rich domains (CRDs) designated 1 through 4, starting from the NH₂-terminus. Each CRD is about 40 amino acids long and contains 4 to 6 cysteine residues at positions which are well conserved [Schall et al., supra; Loetscher et al., supra; Smith et al., supra; Nophar et al., supra; Kohno et al., supra]. In TNFR1, the approximate boundaries of the four CRDs are as follows: CRD1—amino acids 14 to about 53; CRD2—amino acids from about 54 to about 97; CRD3—amino acids from about 98 to about 138; CRD4—amino acids from about 139 to about 167. In TNFR2, CRD1 includes amino acids 17 to about 54; CRD2—amino acids from about 55 to about 97; CRD3—amino acids from about 98 to about 140; and CRD4—amino acids from about 141 to about 179 [Banner et al., Cell, 73:431-435 (1993)]. The potential role of the CRDs in ligand binding is also described by Banner et al., supra.

A similar repetitive pattern of CRDs exists in several other cell-surface proteins, including the p75 nerve growth factor receptor (NGFR) [Johnson et al., Cell, 47:545 (1986); Radeke et al., Nature, 325:593 (1987)], the B cell antigen CD40 [Stamenkovic et al., EMBO J., 8:1403 (1989)], the T cell antigen OX40 [Mallet et al., EMBO J., 9:1063 (1990)] and the Fas antigen [Yonehara et al., supra and Itoh et al., supra]. CRDs are also found in the soluble TNFR (sTNFR) -like T2 proteins of the Shope and myxoma poxviruses [Upton et al., Virology, 160:20-29 (1987); Smith et al., Biochem. Biophys. Res. Commun., 176:335 (1991); Upton et al., Virology, 184:370 (1991)]. Optimal alignment of these sequences indicates that the positions of the cysteine residues are well conserved. These receptors are sometimes collectively referred to as members of the TNF/NGF receptor superfamily. Recent studies on p75NGFR showed that the deletion of CRD1 [Welcher, A.A. et al., Proc. Natl. Acad. Sci. USA, 88:159-163 (1991)] or a 5-amino acid insertion in this domain [Yan, H. and Chao, M. V., J. Biol. Chem., 266:12099-12104 (1991)] had little or no effect on NGF binding [Yan, H. and Chao, M. V., supra]. p75 NGFR contains a proline-rich stretch of about 60 amino acids, between its CRD4 and transmembrane region, which is not involved in NGF binding [Peetre, C. et al., Eur. J. Hematol., 41:414-419 (1988); Seckinger, P. et al., J. Biol. Chem., 264:11966-11973 (1989); Yan, H. and Chao, M. V., supra]. A similar proline-rich region is found in TNFR2 but not in TNFR1.

Itoh et al. disclose that the Apo-1 receptor can signal an apoptotic cell death similar to that signaled by the 55-kDa TNFR1 [Itoh et al., supra]. Expression of the Apo-1 antigen has also been reported to be down-regulated along with that of TNFR1 when cells are treated with either TNF-α or anti-Apo-1 mouse monoclonal antibody [Krammer et al., supra; Nagata et al., supra]. Accordingly, some investigators have hypothesized that cell lines that co-express both Apo-1 and TNFR1 receptors may mediate cell killing through common signaling pathways [Id.].

The TNF family ligands identified to date, with the exception of lymphotoxin-α, are type II transmembrane proteins, whose C-terminus is extracellular. In contrast, the receptors in the TNF receptor (TNFR) family identified to date are type I transmembrane proteins. In both the TNF ligand and receptor families, however, homology identified between family members has been found mainly in the extracellular domain (“ECD”). Several of the TNF family cytokines, including TNF-α, Apo-1 ligand and CD40 ligand, are cleaved proteolytically at the cell surface; the resulting protein in each case typically forms a homotrimeric molecule that functions as a soluble cytokine. TNF receptor family proteins are also usually cleaved proteolytically to release soluble receptor ECDs that can function as inhibitors of the cognate cytokines.

Two of the TNFR family members, TNFRl and Fas/Apol (CD95), can activate apoptotic cell death [Chinnaiyan and Dixit, Current Biology, 6:555-562 (1996); Fraser and Evan, Cell; 85:781-784 (1996)]. TNFR1 is also known to mediate activation of the transcription factor, NF-KB [Tartaglia et al., Cell, 74:845-853 (1993); Hsu et al., Cell, 84:299-308 (1996)]. In addition to some ECD homology, these two receptors share homology in their intracellular domain (ICD) in an oligomerization interface known as the death domain [Tartaglia et al., supra]. Death domains are also found in several metazoan proteins that regulate apoptosis, namely, the Drosophila protein, Reaper, and the mammalian proteins referred to as FADD/MORT1, TRADD, and RIP [Cleaveland and Ihle, Cell, 81:479-482 (1995)]. Using the yeast-two hybrid system, Raven et al. report the identification of protein, wsl-1, which binds to the TNFR1 death domain [Raven et al., Programmed Cell Death Meeting, Sep. 20-24, 1995, Abstract at page 127; Raven et al., European Cytokine Network, 7:Abstr. 82 at page 210 (April-June 1996)]. The wsl-1 protein is described as being homologous to TNFR1 (48% identity) and having a restricted tissue distribution. According to Raven et al., the tissue distribution of wsl-l is significantly different from the TNFR1 binding protein, TRADD.

Upon ligand binding and receptor clustering, TNFR1 and CD95 are believed to recruit FADD into a death-inducing signalling complex. CD95 purportedly binds FADD directly, while TNFR1 binds FADD indirectly via TRADD [Chinnaiyan et al., Cell, 81:505-512 (1995); Boldin et al., J. Biol. Chem., 270:387-391 (1995); Hsu et al., supra; Chinnaiyan et al., J. Biol. Chem., 271:4961-4965 (1996)]. It has been reported that FADD serves as an adaptor 35 protein which recruits the thiol protease MACHα/FLICE into the death signalling complex [Boldin et al., Cell, 85:803-815 (1996); Muzio et al., Cell, 85:817-827 (1996)]. MACHα/FLICE appears to be the trigger that sets off a cascade of apoptotic proteases, including the interleukin-1 β converting enzyme (ICE) and CPP32/Yama, which may execute some critical aspects of the cell death programme [Fraser and Evan, supra].

It was recently disclosed that programmed cell death involves the activity of members of a family of cysteine proteases related to the C. elegans cell death gene, ced-3, and to the mammalian IL-1-converting enzyme, ICE. The activity of the ICE and CPP32/Yama proteases can be inhibited by the product of the cowpox virus gene, crmA [Ray et al., Cell, 69:597-604 (1992); Tewari et al., Cell, 81:801-809 (1995)]. Recent studies show that CrmA can inhibit TNFR1- and CD95-induced cell death [Enari et al., Nature, 375:78-81 (1995); Tewari et al., J. Biol. Chem., 270:3255-3260 (1995)].

As reviewed recently by Tewari et al., TNFR1, TNFR2 and CD40 modulate the expression of proinflammatory and costimulatory cytokines, cytokine receptors, and cell adhesion molecules through activation of the transcription factor, NF-KB [Tewari et al., Curr.

Op. Genet. Develop., 6:39-44 (1996)]. NF-KB is the prototype of a family of dimeric transcription factors whose subunits contain conserved Rel regions [Verma et al., Genes Develop., 9:2723-2735 (1996); Baldwin, Ann. Rev. Immunol., 14:649-681 (1996)]. In its latent form, NF-KB is complexed with members of the IKB inhibitor family; upon inactivation of the IKB in response to certain stimuli, released NF-KB translocates to the nucleus where it binds to specific DNA sequences and activates gene transcription. TNFR proteins may also regulate the AP-1 transcription factor family [Karin, J. Biol. Chem., 270:16483-16486 (1995)]. AP-1 represents a separate family of dimeric transcriptional activators composed of members of the Fos and Jun protein families [Karin, supra]. AP-1 activation is believed to be mediated by immediate-early induction of fos and jun through the mitogen-activated protein kinases ERK and JNK (Jun N-terminal kinase; also known as stress-activated protein kinase, SAPK), as well as by JNK-dependent phosphorylation of Jun proteins [Karin, supra; Kyriakis et al., J. Biol. Chem., 271:24313-24316 (1996)]. Transcriptional regulation by TNFR family members is mediated primarily by members of the TNF receptor associated factor (TRAF) family [Rothe et al., Cell, 78:681-692 (1994); Hsu et al., Cell, 84:299-308 (1996); Liu et al., Cell, 87:565-576 (1996)].

For a review of the TNF family of cytokines and their receptors, see Gruss and Dower, supra.

### SUMMARY OF THE INVENTION

Applicants have identified cDNA clones that encode novel polypeptides, designated in the present application as “Apo-3.” The Apo-3 polypeptide has surprisingly been found to stimulate or induce apoptotic activity in mammalian cells. It is believed that Apo-3 is a member of the TNFR family; full-length native sequence human Apo-3 polypeptide exhibits some similarities to some known TNFRs, including TNFRl and CD95. In particular, full-length native sequence human Apo-3 exhibits similarity to the TNFR family in its extracellular cysteine-rich repeats and resembles TNFR1 and CD95 in that it contains a cytoplasmic death domain sequence.

Applicants have also identified cDNA clones that encode a polypeptide, designated “Apo-2 ligand inhibitor” or “Apo-2LI”. Although not being bound to any particular theory, it is presently believed that Apo-2LI comprising amino acid residues 1 to 181 of FIG. 1 (SEQ ID NO:1) [and which correspond to amino acid residues 1 to 181 of the sequence of FIG. 4 (SEQ ID NO:6)] may be a soluble, truncated or secreted form of Apo-3.

In one embodiment, the invention provides isolated Apo-2LI. In particular, the invention provides isolated native sequence Apo-2LI, which in one embodiment, includes an amino acid sequence comprising residues 1 to 181 of FIG. 1 (SEQ ID NO:1). In other embodiments, the isolated Apo-2LI comprises one or more cysteine-rich domains of the sequence of FIG. 1, or comprises biologically active polypeptides comprising at least about 80% identity with native sequence Apo-2LI shown in FIG. 1 (SEQ ID NO:1).

In another embodiment, the invention provides chimeric molecules comprising Apo-2LI fused to another, heterologous polypeptide or amino acid sequence. An example of such a chimeric molecule comprises an Apo-2LI amino acid sequence fused to an immunoglobulin constant domain sequence.

In another embodiment, the invention provides an isolated nucleic acid molecule encoding Apo-2LI. In one aspect, the nucleic acid molecule is RNA or DNA that encodes an Apo-2LI or is complementary to a nucleic acid sequence encoding such Apo-2LI, and remains stably bound to it under stringent conditions. In one embodiment, the nucleic acid sequence is selected from:

(a) the coding region of the nucleic acid sequence of FIG. 1 that codes for residue 1 to residue 181 (i.e., nucleotides 377 through 919; also provided in SEQ ID NO:5), inclusive; or

(b) a sequence corresponding to the sequence of (a) within the scope of degeneracy of the genetic code.

In a further embodiment, the invention provides a replicable vector comprising the nucleic acid molecule encoding the Apo-2LI operably linked to control sequences recognized by a host cell transfected or transformed with the vector. A host cell comprising the vector or the nucleic acid molecule is also provided. A method of producing Apo-2LI which comprises culturing a host cell comprising the nucleic acid molecule and recovering the protein from the host cell culture is further provided.

In another embodiment, the invention provides an antibody which binds to Apo-2LI.

In another embodiment, the invention provides isolated Apo-3 polypeptide. In particular, the invention provides isolated native sequence Apo-3 polypeptide, which in one embodiment, includes an amino acid sequence comprising residues 1 to 417 of FIG. 4 (SEQ ID NO:6). In other embodiments, the isolated Apo-3 polypeptide comprises at least about 80% identity with native sequence Apo-3 polypeptide comprising residues 1 to 417 of FIG. 4 (SEQ ID NO:6).

In another embodiment, the invention provides an isolated extracellular domain sequence of Apo-3 polypeptide. The isolated extracellular domain sequence preferably comprises residues 1 to 198 of FIG. 4 (SEQ ID NO:6).

In another embodiment, the invention provides an isolated death domain sequence of Apo-3 polypeptide. The isolated death domain sequence preferably comprises residues 338 to 417 of FIG. 4 (SEQ ID NO:6).

In another embodiment, the invention provides chimeric molecules comprising Apo-3 polypeptide fused to a heterologous polypeptide or amino acid sequence. An example of such a chimeric molecule comprises an Apo-3 fused to an immunoglobulin sequence. Another example comprises an extracellular domain sequence of Apo-3 fused to a heterologous polypeptide or amino acid sequence, such as an immunoglobulin sequence.

In another embodiment, the invention provides an isolated nucleic acid molecule encoding Apo-3 polypeptide. In one aspect, the nucleic acid molecule is RNA or DNA that encodes an Apo-3 polypeptide or a particular domain of Apo-3, or is complementary to such encoding nucleic acid sequence, and remains stably bound to it under stringent conditions. In one embodiment, the nucleic acid sequence is selected from:

(a) the coding region of the nucleic acid sequence of FIG. 4 (SEQ ID NO:9) that codes for residue 1 to residue 417 (i.e., nucleotides 89-91 through 1337-1339), inclusive; or

(b) the coding region of the nucleic acid sequence of FIG. 4 (SEQ ID NO:9) that codes for residue 1 to residue 198 (i.e., nucleotides 89-91 through 680-682), inclusive;

(c) the coding region of the nucleic acid sequence of FIG. 4 (SEQ ID NO:9) that codes for residue 338 to residue 417 (i.e., nucleotides 1100-1102 through 1337-1339), inclusive; or

(d) a sequence corresponding to the sequence of (a), (b) or (c) within the scope of degeneracy of the genetic code.

In a further embodiment, the invention provides a vector comprising the nucleic acid molecule encoding the Apo-3 polypeptide or particular domain of Apo-3. A host cell comprising the vector or the nucleic acid molecule is also provided. A method of producing Apo-3 is further provided.

In another embodiment, the invention provides an antibody which binds to Apo-3.

In another embodiment, the invention provides non-human, transgenic or knock-out animals.

A further embodiment of the invention provides articles of manufacture and kits.

### BRIEF DESCRIPTION OF THE DRAWINGS

FIG. 1 shows the nucleotide sequence of human Apo-2LI cDNA and its derived amino acid sequence.

FIG. 2 shows an alignment of the amino acid sequence encoded by clone 18.1 of Apo-2LI with extracellular regions of other members of the human TNF receptor family.

FIG. 3 shows a silver-stained gel of a protein A purified Apo-2LI immunoadhesin analyzed under non-reducing (lanes 3-5) or reducing (lanes 7-9) conditions.

FIG. 4 shows the nucleotide sequence of native sequence human Apo-3 cDNA and its derived amino acid sequence. The putative signal sequence and transmembrane domain are underlined, the death domain sequence is boxed, and the potential N-linked glycosylation sites are marked with an asterisk. Also boxed is the alanine residue which was present in the fetal lung but not in the fetal heart cDNA clone (discussed in Example 4 below).

FIG. 5 shows an alignment and comparison of the ECD sequences of native sequence human Apo-3, TNFR1 and CD95.

FIG. 6 shows an alignment and comparison of the death domain sequences of native sequence human Apo-3, TNFR1, CD95, FADD, TRADD, RIP and Drosophila Reaper.

FIG. 7 shows a schematic alignment of Apo-3, Apo-2LI, TNFR1, and Fas/Apo-1. CRD, cysteine-rich domains; TM, transmembrane domain; DD, death domain.

FIG. 8 shows ectopic expression of Apo-3 in HEK 293 cells. Cells were transfected with pRK5-Apo-3 plus pRK5 (5 μg each) (lane 1); pRK5 alone (10 μg) (lane 2); or pRKS-Apo-3 plus pRK5-CrmA (5 μg each) (lane 3). Cells were metabolically labeled with ³⁵S-Met and ³⁵S-Cys. Cell lysates were then analyzed by radioimmunoprecipitation using mouse anti-Apo-3 antiserum. The molecular weight standards are shown on the left in kDa.

FIGS. 9a-j illustrate the induction of apoptosis by ectopic expression of Apo-3 in HEK 293 cells. Apoptosis was examined 36 hours after transfection, by morphological analysis (FIGS. 9a-d); by FACS analysis (FIGS. 9 e-i); and by DNA laddering (FIG. 9 j). Cells were transfected with pRK5 alone (10 μg) (FIGS. 9 a; e; j, lane 1); pRK5 plus pRK5-Apo-3 (5 μg each) (FIGS. 9 b; f; j, lane 2); PRK5 plus pRK5-CrmA (5 μg each) (FIGS. 9 c; g; j, lane 3); or pRK5-Apo-3 plus pRK5-CrmA (5 μg each) (FIGS. 9 d; h; j, lane 4). Cells in FIGS. 9 a-d were photographed at 400X magnification using Hoffmann optics-based light microscopy. As measured by the total number of annexin V-positive cells, the percent apoptosis in FIGS. 9 e-h, respectively, was 37%, 66%, 36% and 26%. Cells in FIG. 9 i were transfected with the indicated amount of pRK5-Apo-3 or pRK5-TNFR1 and the appropriate amount of pRK5 plasmid to bring the total amount of DNA to 20 μg.

FIG. 10 shows activation of NF-KB by ectopic expression of Apo-3. HEK 293 cells were transfected with 10 μg pRK5 (lanes 1, 4, 7); pRK5-Apo-3 (lanes 2, 5, 7); or pRK5-TNFR1 (lanes 3, 6, 9).

Nuclear extracts were prepared 36 hours later and reacted with an irrelevant ³²P-labelled oligonucleotide probe (lanes 1-3); or with a ³²P-labelled NF-KB-specific probe alone (lanes 4-6) or in the presence of 50-fold excess unlabelled oligonucleotide of the same sequence (lanes 7-9).

FIG. 11 shows activation of Jun N-terminal kinase (JNK) by ectopic expression of Apo-3. HEK 293 cells were transfected with 10 μg pRK5, pRK5-TNFR1, or pRK5-Apo-3 as indicated. 36 hours later, JNK activity in cell extracts was determined using a JNK/SAPK activation kit, which measures JNK activity by analyzing phosphorylation of c-Jun.

FIG. 12 illustrates expression of Apo-3 mRNA in human tissues as determined by Northern blot hybridization. In the left hand panel are shown fetal brain (1); lung (2); liver (3); kidney (4). In the right hand panel are shown adult spleen (1); thymus (2); prostate (3); testis (4); ovary (5); small intestine (6);

colon (7); and peripheral blood lymphocytes (8). The sizes of the molecular weight standards are shown on the left in kb.

### DETAILED DESCRIPTION OF THE PREFERRED EMBODIMENTS

I. Definitions The terms “Apo-3 polypeptide” and “Apo-3” when used herein encompass native sequence Apo-3 and Apo-3 variants (each of which is defined herein). These terms encompass Apo-3 from a variety of mammals, including humans. The Apo-3 may be isolated from a variety of sources, such as from human tissue types or from another source, or prepared by recombinant or synthetic methods.

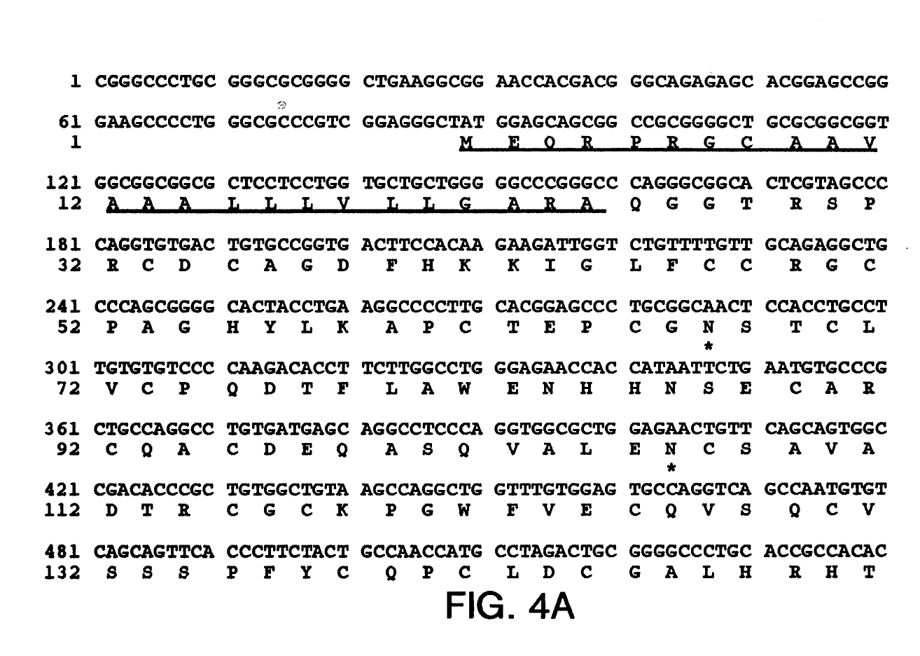

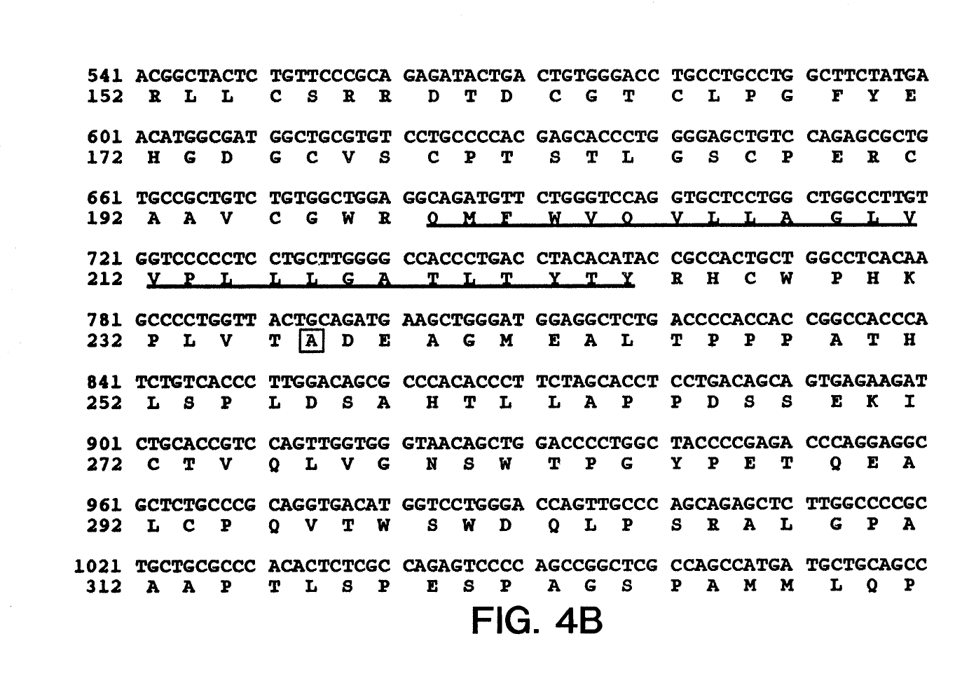

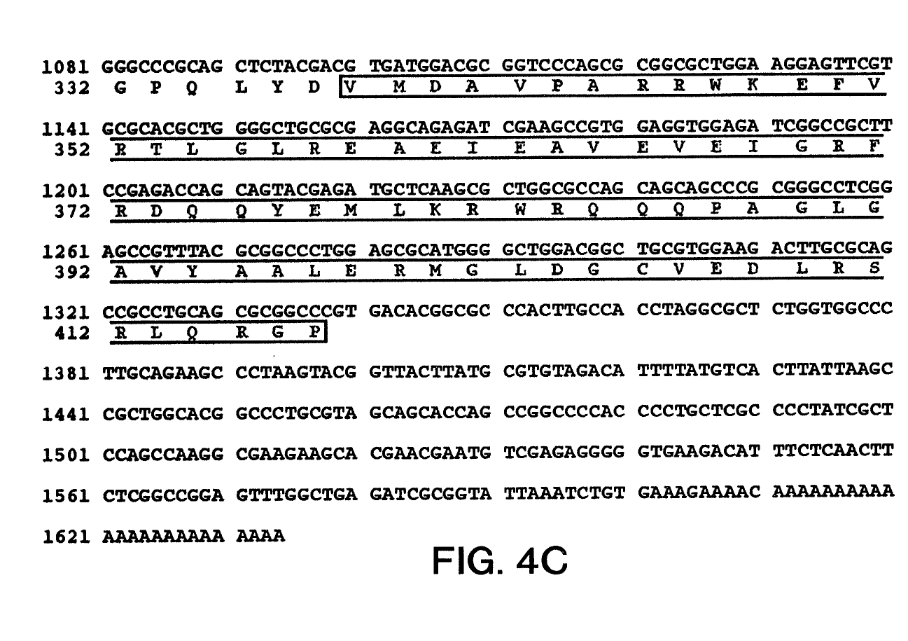

A “native sequence Apo-3” comprises a polypeptide having the same amino acid sequence as an Apo-3 derived from nature. Thus, a native sequence Apo-3 can have the amino acid sequence of naturally-occurring Apo-3 from any mammal. Such native sequence Apo-3 can be isolated from nature or can be produced by recombinant or synthetic means. The term “native sequence Apo-3” specifically encompasses naturally-occurring truncated or secreted forms of the Apo-3 (e.g., an extracellular domain sequence), naturally-occurring variant forms (e.g., alternatively spliced forms) and naturally-occurring allelic variants of the Apo-3. A naturally-occurring variant form of the Apo-3 includes an Apo-3 having an amino acid deletion at residue 236 in the amino acid sequence shown in FIG. 4 (SEQ ID NO:6). In one embodiment of the invention, the native sequence Apo-3 is a mature or full-length native sequence Apo-3 comprising the amino acid sequence of SEQ ID NO:6. The present definition of native sequence Apo-3 excludes known EST sequences, such as GenBank W71984.

“Apo-3 variant” means a biologically active Apo-3 as defined below having less than 100% sequence identity with Apo-3 having the deduced amino acid sequence shown in FIG. 4 (SEQ ID NO:6) for a full-length native sequence human Apo-3. Such Apo-3 variants include, for instance, Apo-3 polypeptides wherein one or more amino acid residues are added at the N- or C-terminus of, or within, the sequence of SEQ ID NO:6; from about one to 24 amino acid residues are deleted (including a single amino acid deletion at residue 236 in the amino acid sequence shown in FIG. 4 (SEQ ID NO:6), or optionally substituted by one or more amino acid residues; and derivatives thereof, wherein an amino acid residue has been covalently modified so that the resulting product has a non-naturally occurring amino acid. ordinarily, an Apo-3 variant will have at least about 80% sequence identity, more preferably at least about 90% sequence identity, and even more preferably at least about 95% sequence identity with the sequence of FIG. 4 (SEQ ID NO:6). The present definition of Apo-3 variant excludes known EST sequences, such as GenBank W71984.

The terms “Apo-2 ligand inhibitor polypeptide” and “Apo-2LI” when used herein encompass native sequence Apo-2LI and Apo-2LI variants (each of which is defined herein). These terms encompass Apo-2LI from a variety of mammals, including humans. The Apo-2LI may be isolated from a variety of sources, such as from human tissue types or from another source, or prepared by recombinant or synthetic methods.

A “native sequence Apo-2LI” comprises a polypeptide having the same amino acid sequence as an Apo-2LI derived from nature. Thus, a native sequence Apo-2LI can have the amino acid sequence of naturally-occurring Apo-2LI from any mammal. Such native sequence Apo-2LI can be isolated from nature or can be produced by recombinant or synthetic means. The term “native sequence Apo-2LI” specifically encompasses naturally-occurring truncated forms, naturally-occurring variant forms (e.g., alternatively spliced forms) and naturally-occurring allelic variants. In one embodiment of the invention, the native sequence Apo-2LI comprises the amino acid sequence of SEQ ID NO:1. The present definition of native sequence Apo-2LI excludes known EST sequences, such as GenBank H41522, H46424, H46211, H46374, H46662, H41851, H49675, H22502, H46378 and H19739.

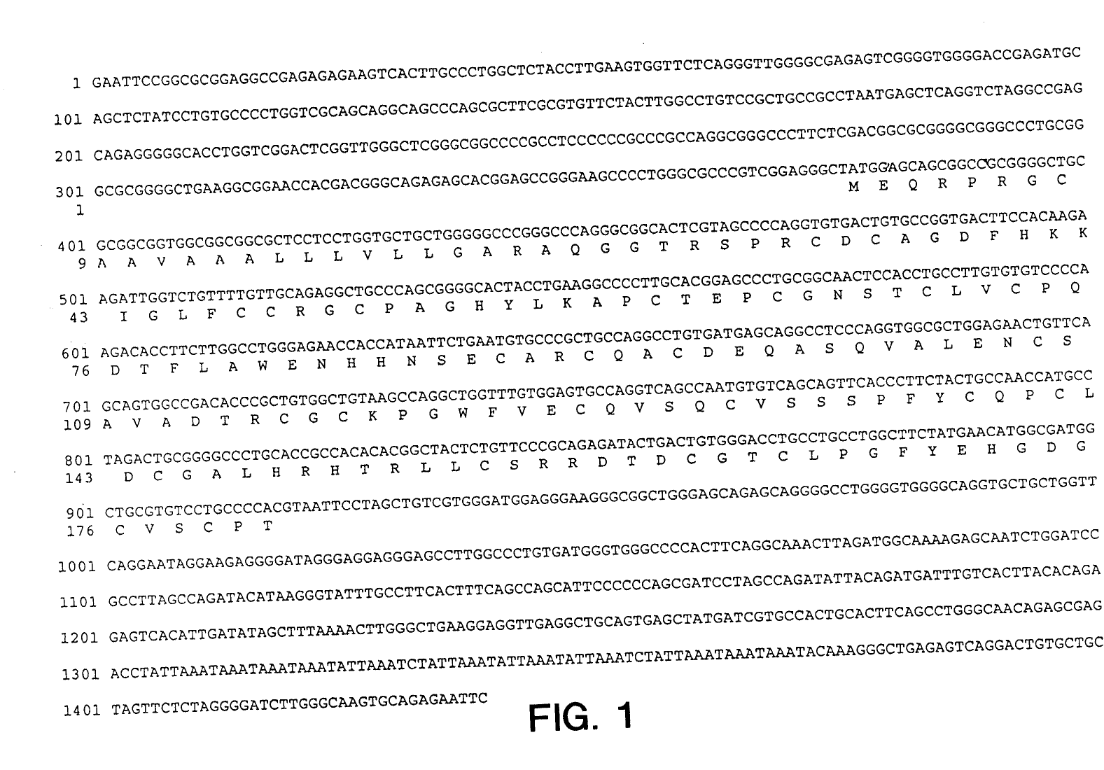

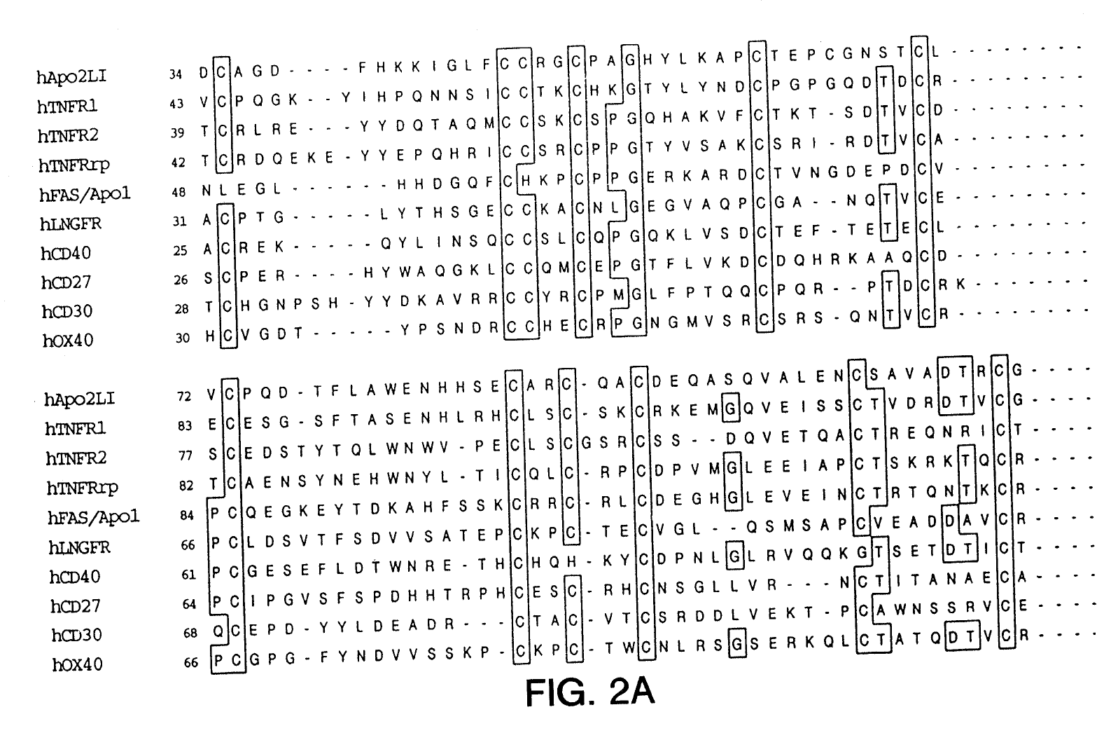

“Apo-2LI variant” means a biologically active Apo-2LI as defined below having less than 100% sequence identity with Apo-2LI having the deduced amino acid sequence shown in FIG. 1 (SEQ ID NO:1). Such Apo-2LI variants include, for instance, Apo-2LI polypeptides wherein one or more amino acid residues are added at the N- or C-terminus of, or within, the sequence of SEQ ID NO:1; wherein one or more amino acid residues are deleted, or optionally substituted by one or more amino acid residues; and derivatives thereof, wherein an amino acid residue has been covalently modified so that the resulting product has a non-naturally occurring amino acid. Optionally, the Apo-2LI includes one or more cysteine-rich domains, and preferably includes one or more cysteine-rich domains comprising amino acids 34 to 71, amino acids 72 to 115, amino acids 116 to 163, or amino acids 164 to 181 of FIG. 1. Ordinarily, an Apo-2LI variant will have at least about 80% sequence identity, more preferably at least about 90% sequence identity, and even more preferably at least about 95% sequence identity with the sequence of FIG. 1 (SEQ ID NO:1). The present definition of Apo-2LI variant excludes known EST sequences, such as GenBank H41522, H46424, H46211, H46374, H46662, H41851, H49675, H22502, H46378 and H19739.

The term “epitope tagged” when used herein refers to a chimeric polypeptide comprising at least one of the Apo-3 or Apo-2LI polypeptides disclosed herein, or a portion thereof, fused to a “tag polypeptide”. The tag polypeptide has enough residues to provide an epitope against which an antibody can be made, yet is short enough such that it does not interfere with activity of the Apo-3 or Apo-2LI. The tag polypeptide preferably also is fairly unique so that the antibody does not substantially cross-react with other epitopes. Suitable tag polypeptides generally have at least six amino acid residues and usually between about 8 to about 50 amino acid residues (preferably, between about 10 to about 20 residues).

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the Apo-3 or Apo-2LI natural environment will not be present. Ordinarily, however, isolated polypeptide will be prepared by at least one purification step.

An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells. However, an isolated Apo-3 nucleic acid molecule, for instance, includes Apo-3 nucleic acid molecules contained in cells that ordinarily express Apo-3 where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers single monoclonal antibodies (including agonist, antagonist, and neutralizing antibodies) and antibody compositions with polyepitopic specificity.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The monoclonal antibodies herein include hybrid and recombinant antibodies produced by splicing a variable (including hypervariable) domain of an anti-Apo-3 antibody or anti-Apo-2LI antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity. See, e.g. U.S. Pat. No. 4,816,567 and Mage et al., in Monoclonal Antibody Production Techniques and Applications, pp.79-97 (Marcel Dekker, Inc.: New York, 1987).

Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature, 256:495 (1975), or may be made by recombinant DNA methods such as described in U.S. Pat. No. 4,816,567. The “monoclonal antibodies” may also be isolated from phage libraries generated using the techniques described in McCafferty et al., Nature, 348:552-554 (1990), for example.

“Humanized” forms of non-human (e.g. murine) antibodies are specific chimeric immunoglobulins, immunoglobulin chains, or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and 5 optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human 10 immunoglobulin consensus sequence. The humanized antibody optimally also will comprise at least a portion of an immunoglobulin constant region or domain (Fc), typically that of a human immunoglobulin.

“Biologically active” and “desired biological activity” for the purposes herein mean having the ability to modulate apoptosis (either in an agonistic manner by inducing or stimulating apoptosis, or in an antagonistic manner by reducing or inhibiting apoptosis) in at least one type of mammalian cell in vivo or ex vivo.

The terms “apoptosis” and “apoptotic activity” are used in a broad sense and refer to the orderly or controlled form of cell death in mammals that is typically accompanied by one or more characteristic cell changes, including condensation of cytoplasm, loss of plasma membrane microvilli, segmentation of the nucleus, degradation of chromosomal DNA or loss of mitochondrial function. This activity can be determined and measured, for instance, by cell viability assays, FACS analysis or DNA electrophoresis, all of which are known in the art.

The terms “treating,” “treatment,” and “therapy” as used herein refer to curative therapy, prophylactic therapy, and preventative therapy.

The term “mammal” as used herein refers to any mammal classified as a mammal, including humans, cows, horses, dogs and cats. In a preferred embodiment of the invention, the mammal is a human.

II. Compositions and Methods of the Invention

Applicants have identified and isolated various polypeptides involved in mammalian cell apoptosis. In particular, Applicants have identified and isolated various Apo-3 polypeptides and forms thereof, referred to herein as Apo-2LI. The properties and characteristics of some of these Apo-3 and Apo-2LI polypeptides, particularly human Apo-3 and human Apo-2LI, are described in further detail in the Examples below. Based upon the properties and characteristics of the Apo-3 and Apo-2LI polypeptides disclosed herein, it is Applicants' present belief that Apo-3 is a member of the TNFR family.

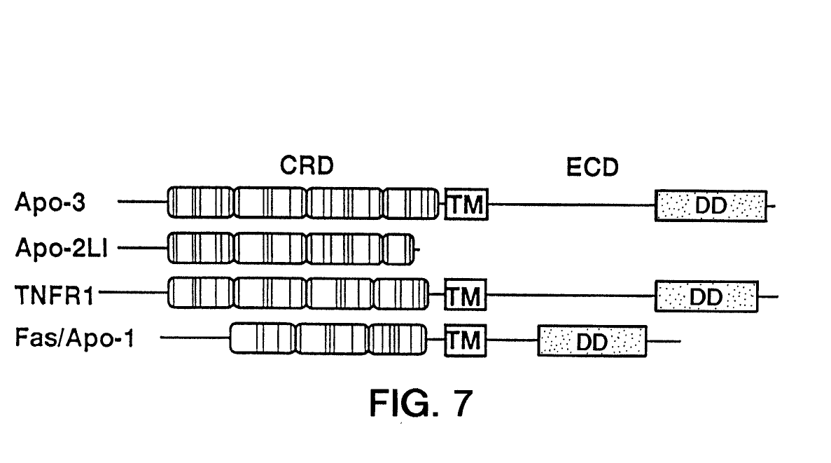

As discussed in Example 4 below, a native sequence human Apo-3 polypeptide was identified. The predicted polypeptide precursor is 417 amino acids long (see FIG. 4). Hydropathy analysis (not shown) suggested the presence of a signal sequence (residues 1-24), followed by an extracellular domain (residues 25-198), a transmembrane domain (residues 199-224), and an intracellular domain (residues 225-417) (FIG. 4; SEQ ID NO:6) The schematic diagram shown in FIG. 7 illustrates such domains, as well as the cysteine-rich domains.

As discussed in Example 1, Applicants also identified and isolated a polypeptide referred to herein as “Apo-2LI.” The predicted amino acid sequence of human Apo-2LI contains 181 amino acids (as shown in FIG. 1). It is presently believed that Apo-2LI comprising amino acid residues 1 to 181 of FIG. 1 may be a soluble, truncated or secreted form of Apo-3. The human Apo-2LI having amino acids 1 to 181 of FIG. 1 is substantially homologous (i.e., having at least 80% identity) to the extracellular sequence of native sequence human Apo-3 (amino acid residues 1 to 198, as shown in FIG. 4), and it is presently believed that such Apo-2LI (at least in monomeric form) is a functional equivalent to the Apo-3.

A description follows as to how the polypeptides of the invention, as well as chimeric molecules and antibodies, may be prepared. It is contemplated that the methods and materials described below (and in the Examples herein) may be employed to prepare Apo-2LI, Apo-2LI chimeric molecules and anti-Apo-2LI antibodies, as well as Apo-3 polypeptides, Apo-3 chimeric molecules and anti-Apo-3 antibodies.

A. Preparation of Polypeptides and Nucleic Acids

The description below relates primarily to production of the polypeptides by culturing cells transformed or transfected with a vector containing Apo-3 or Apo-2LI nucleic acid. It is of course, contemplated that alternative methods, which are well known in the art, may be employed to prepare the polypeptides.

1. Isolation of Encoding DNA

The DNA encoding the polypeptides of the invention may be obtained from any cDNA library prepared from tissue believed to possess the polypeptide mRNA and to express it at a detectable level. Accordingly, human Apo-2LI DNA can be conveniently obtained from a cDNA library prepared from human tissues, such as the bacteriophage library of human thymus cDNA described in Example 1. The Apo-2LI-encoding gene may also be obtained from a genomic library or by oligonucleotide synthesis. Human Apo-3 DNA can be conveniently obtained from a cDNA library prepared from human tissues, such as the bacteriophage libraries of human fetal heart and lung cDNA described in Example 4. The Apo-3-encoding gene may also be obtained from a genomic library or by oligonucleotide synthesis.

Libraries can be screened with probes (such as antibodies or oligonucleotides of at least about 20-80 bases) designed to identify the gene of interest or the protein encoded by it. Examples of oligonucleotide probes are provided in Examples 1 and 4. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding, for instance, Apo-3 or Apo-2LI is to use PCR methodology [Sambrook et al., supra; Dieffenbach et al., PCR Primer:A Laboratory Manual (Cold Spring Harbor Laboratory Press, 1995)].

A preferred method of screening employs selected oligonucleotide sequences to screen cDNA libraries from various human tissues. Examples 1 and 4 below describe techniques for screening CDNA libraries with different oligonucleotide probes. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling.

Nucleic acid having all the protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

Apo-3 or Apo-2LI variants can be prepared by introducing appropriate nucleotide changes into the DNA of such polypeptides, or by synthesis of the desired polypeptide. Any combination of insertion, substitution, and/or deletion can be made to arrive at the final construct, provided that the final construct possesses the desired activity as defined herein. Those skilled in the art will appreciate that amino acid changes may alter post-translational processes of the Apo-3, such as changing the number or position of glycosylation sites or altering the membrane anchoring characteristics.

Variations in the native sequence Apo-3 or Apo-2LI as described above can be made using any of the techniques and guidelines for conservative and non-conservative mutations set forth in U.S. Pat. No. 5,364,934. These include oligonucleotide-mediated (site-directed) mutagenesis, alanine scanning, and PCR mutagenesis.

2. Insertion of Nucleic Acid into A Replicable Vector

The nucleic acid (e.g., cDNA or genomic DNA) encoding Apo-3 or Apo-2LI may be inserted into a replicable vector for further cloning (amplification of the DNA) or for expression. Various vectors are publicly available. The vector components generally include, but are not limited to, one or more of the following: a signal sequence, an origin of replication, one or more marker genes, an enhancer element, a promoter, and a transcription termination sequence, each of which is described below.

(i) Signal Sequence Component

The polypeptides of the invention may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the DNA that is inserted into the vector. The heterologous signal sequence selected preferably is one that is recognized and processed (i.e., cleaved by a signal peptidase) by the host cell. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces a-factor leaders, the latter described in U.S. Pat. No. 5,010,182), or acid phosphatase leader, the C. albicans glucoamylase leader (EP 362,179 published Apr. 4, 1990), or the signal described in WO 90/13646 published Nov. 15, 1990. In mammalian cell expression the native Apo-3 or Apo-2LI presequence that normally directs insertion of the polypeptide in the cell membrane of human cells in vivo is satisfactory, although other mammalian signal sequences may be used to direct secretion of the protein, such as signal sequences from secreted polypeptides of the same or related species, as well as viral secretory leaders, for example, the herpes simplex glycoprotein D signal.

The DNA for such precursor region is preferably ligated in reading frame to DNA encoding the polypeptide.

(ii) Origin of Replication Component

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used because it contains the early promoter).

Most expression vectors are “shuttle” vectors, i.e., they are capable of replication in at least one class of organisms but can be transfected into another organism for expression. For example, a vector is cloned in E. coli and then the same vector is transfected into yeast or mammalian cells for expression even though it is not capable of replicating independently of the host cell chromosome.

DNA may also be amplified by insertion into the host genome. This is readily accomplished using Bacillus species as hosts, for example, by including in the vector a DNA sequence that is complementary to a sequence found in Bacillus genomic DNA. Transfection of Bacillus with this vector results in homologous recombination with the genome and insertion of the polypeptide's DNA. However, the recovery of genomic DNA encoding Apo-3 or Apo-2LI is more complex than that of an exogenously replicated vector because restriction enzyme digestion is required to excise the DNA.

(iii) Selection Gene Component

Expression and cloning vectors typically contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin [Southern et al., J. Molec. Appl. Genet., 1:327 (1982)], mycophenolic acid (Mulligan et al., Science, 209:1422 (1980)] or hygromycin [Sugden et al., Mol. Cell. Biol., 5:410-413 (1985)]. The three examples given above employ bacterial genes under eukaryotic control to convey resistance to the appropriate drug G418 or neomycin (geneticin), xgpt (mycophenolic acid), or hygromycin, respectively.

Another example of suitable selectable markers for mammalian cells are those that enable the identification of cells competent to take up the Apo-3 or Apo-2LI nucleic acid, such as DHFR or thymidine kinase. The mammalian cell transformants are placed under selection pressure that only the transformants are uniquely adapted to survive by virtue of having taken up the marker. Selection pressure is imposed by culturing the transformants under conditions in which the concentration of selection agent in the medium is successively changed, thereby leading to amplification of both the selection gene and the DNA that encodes the polypeptide. Amplification is the process by which genes in greater demand for the production of a protein critical for growth are reiterated in tandem within the chromosomes of successive generations of recombinant cells. Increased quantities of Apo-3 or Apo-2LI are synthesized from the amplified DNA. Other examples of amplifiable genes include metallothionein-I and -II, adenosine deaminase, and ornithine decarboxylase.

Cells transformed with the DHFR selection gene may first be identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity, prepared and propagated as described by Urlaub et al., Proc. Natl. Acad. Sci. USA, 77:4216 (1980). The transformed cells are then exposed to increased levels of methotrexate. This leads to the synthesis of multiple copies of the DHFR gene, and, concomitantly, multiple copies of other DNA comprising the expression vectors, such as the DNA encoding the Apo-3 or Apo-2LI. This amplification technique can be used with any otherwise suitable host, e.g., ATCC No. CCL61 CHO-K1, notwithstanding the presence of endogenous DHFR if, for example, a mutant DHFR gene that is highly resistant to Mtx is employed (EP 117,060).

Alternatively, host cells (particularly wild-type hosts that contain endogenous DHFR) transformed or co-transformed with DNA sequences encoding Apo-3 or Apo-2LI, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trpl gene present in the yeast plasmid YRp7 [Stinchcomb et al., Nature, 282:39 (1979); Kingsman et al., Gene, 7:141 (1979); Tschemper et al., Gene, 10:157 (1980)]. The trpl gene provides a selection marker for a mutant strain of yeast lacking the ability to grow in tryptophan, for example, ATCC No. 44076 or PEP4-1 [Jones, Genetics, 85:12 (1977)]. The presence of the trpl lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts [Bianchi et al., Curr. Genet., 12:185 (1987)]. More recently, an expression system for large-scale production of recombinant calf chymosin was reported for K. lactis [Van den Berg, Bio/Technology, 8:135 (1990)]. Stable multi-copy expression vectors for secretion of mature recombinant human serum albumin by industrial strains of Kluyveromyces have also been disclosed [Fleer et al., Bio/Technology, 9:968-975 (1991)].

(iv) Promoter Component

Expression and cloning vectors usually contain a promoter that is recognized by the host organism and is operably linked to the Apo-3 or Apo-2LI nucleic acid sequence. Promoters are untranslated sequences located upstream (5′) to the start codon of a structural gene (generally within about 100 to 1000 bp) that control the transcription and translation of particular nucleic acid sequence, such as the Apo-3 or Apo-2LI nucleic acid sequence, to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to Apo-3 or Apo-2LI encoding DNA by removing the promoter from the source DNA by restriction enzyme digestion and inserting the isolated promoter sequence into the vector. Both the native Apo-3 or Apo-2LI promoter sequence and many heterologous promoters may be used to direct amplification and/or expression of DNA encoding the polypeptide.

Promoters suitable for use with prokaryotic hosts include the β-lactamase and lactose promoter systems [Chang et al., Nature, 275:617-624 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. However, other known bacterial promoters are suitable. Their nucleotide sequences have been published, thereby enabling a skilled worker operably to ligate them to DNA encoding Apo-3 or Apo-2LI [Siebenlist et al., Cell, 20:269 (1980)] using linkers or adaptors to supply any required restriction sites. Promoters for use in bacterial systems also will contain a Shine-Dalgarno (S. D.) sequence operably linked to the DNA.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CXCAAT region where X may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase [Hitzeman et al., J. Biol. Chem., 255:12073-12080 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Transcription from vectors in mammalian host cells is controlled, for example, by promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, and from the promoter normally associated with the Apo-3 or Apo-2LI sequence, provided such promoters are compatible with the host cell systems.

The early and late promoters of the SV40 virus are conveniently obtained as an SV40 restriction fragment that also contains the SV40 viral origin of replication [Fiers et al., Nature, 273:113 (1978); Mulligan and Berg, Science, 209:1422-1427 (1980); Pavlakis et al., Proc. Natl. Acad. Sci. USA, 78:7398-7402 (1981)]. The immediate early promoter of the human cytomegalovirus is conveniently obtained as a HindIII E restriction fragment [Greenaway et al., Gene, 18:355-360 (1982)]. A system for expressing DNA in mammalian hosts using the bovine papilloma virus as a vector is disclosed in U.S. Pat. No. 4,419,446. A modification of this system is described in U.S. Pat. No. 4,601,978 [See also Gray et al., Nature, 295:503-508 (1982) on expressing cDNA encoding immune interferon in monkey cells; Reyes et al., Nature, 297:598-601 (1982) on expression of human β-interferon cDNA in mouse cells under the control of a thymidine kinase promoter from herpes simplex virus; Canaani and Berg, Proc. Natl. Acad. Sci. USA 79:5166-5170 (1982) on expression of the human interferon β1 gene in cultured mouse and rabbit cells; and Gorman et al., Proc. Natl. Acad. Sci. USA, 79:6777-6781 (1982) on expression of bacterial CAT sequences in CV-l monkey kidney cells, chicken embryo fibroblasts, Chinese hamster ovary cells, HeLa cells, and mouse NIH-3T3 cells using the Rous sarcoma virus long terminal repeat as a promoter].

(v) Enhancer Element Component

Transcription of a DNA encoding the Apo-3 or Apo-2LI of this invention by higher eukaryotes may be increased by inserting an enhancer sequence into the vector. Enhancers are cis-acting elements of DNA, usually about from 10 to 300 bp, that act on a promoter to increase its transcription. Enhancers are relatively orientation and position independent, having been found 5′ [Laimins et al., Proc. Natl. Acad. Sci. USA, 78:464-468 (1981)] and 3′ [Lusky et al., Mol. Cell Bio., 3:1108 (1983]) to the transcription unit, within an intron [Banerji et al., Cell, 33:729 (1983)], as well as within the coding sequence itself [Osborne et al., Mol. Cell Bio., 4:1293 (1984)]. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the coding sequence, but is preferably located at a site 5′ from the promoter.

(vi) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding Apo-3 or Apo-2LI.

(vii) Construction and Analysis of Vectors

Construction of suitable vectors containing one or more of the above-listed components employs standard ligation techniques. Isolated plasmids or DNA fragments are cleaved, tailored, and re-ligated in the form desired to generate the plasmids required.

For analysis to confirm correct sequences in plasmids constructed, the ligation mixtures can be used to transform E. Coli K12 strain 294 (ATCC 31,446) and successful transformants selected by ampicillin or tetracycline resistance where appropriate. Plasmids from the transformants are prepared, analyzed by restriction endonuclease digestion, and/or sequenced by the method of Messing et al., Nucleic Acids Res., 9:309 (1981) or by the method of Maxam et al., Methods in Enzymology, 65:499 (1980).

(viii) Transient Expression Vectors

Expression vectors that provide for the transient expression in mammalian cells of DNA encoding Apo-3 or Apo-2LI may be employed. In general, transient expression involves the use of an expression vector that is able to replicate efficiently in a host cell, such that the host cell accumulates many copies of the expression vector and, in turn, synthesizes high levels of a desired polypeptide encoded by the expression vector [Sambrook et al., supra]. Transient expression systems, comprising a suitable expression vector and a host cell, allow for the convenient positive identification of polypeptides encoded by cloned DNAs, as well as for the rapid screening of such polypeptides for desired biological or physiological properties. Thus, transient expression systems are particularly useful in the invention for purposes of identifying Apo-3 variants or Apo-2LI variants.

(ix) Suitable Exemplary Vertebrate Cell Vectors

Other methods, vectors, and host cells suitable for adaptation to the synthesis of Apo-3 or Apo-2LI in recombinant vertebrate cell culture are described in Gething et al., Nature, 293:620-625 (1981); Mantei et al., Nature, 281:40-46 (1979); EP 117,060; and EP 117,058. A particularly useful plasmid for mammalian cell culture expression of Apo-2LI is pRK7 [EP 278,776; also described in Example 1].

3. Selection and Transformation of Host Cells

Suitable host cells for cloning or expressing the DNA in the vectors herein are the prokaryote, yeast, or higher eukaryote cells described above. Suitable prokaryotes for this purpose include but are not limited to eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. Preferably, the host cell should secrete minimal amounts of proteolytic enzymes.

In addition to prokaryotes, eukaryotic microbes such as filamentous fungi or yeast are suitable cloning or expression hosts. Saccharomyces cerevisiae, or common baker's yeast, is the most commonly used among lower eukaryotic host microorganisms. However, a number of other genera, species, and strains are commonly available and useful herein.

Suitable host cells for the expression of glycosylated polypeptides are derived from multicellular organisms. Such host cells are capable of complex processing and glycosylation activities. In principle, any higher eukaryotic cell culture is workable, whether from vertebrate or invertebrate culture. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila inelanogaster (fruitfly), and Bombyx mori have been identified [See, e.g., Luckow et al., Bio/Technology, 6:47-55 (1988); Miller et al., in Genetic Engineering, Setlow et al., eds., Vol. 8 (Plenum Publishing, 1986), pp. 277-279; and Maeda et al., Nature, 315:592-594 (1985)]. A variety of viral strains for transfection are publicly available, e.g., the L-1 variant of Autographa californica NPV and the Bm-5 strain of Bombyx mori NPV.

Plant cell cultures of cotton, corn, potato, soybean, petunia, tomato, and tobacco can be utilized as hosts. Typically, plant cells are transfected by incubation with certain strains of the bacterium Agrobacterium tumefaciens. During incubation of the plant cell culture with A. tumefaciens, the DNA encoding the polypeptide can be transferred to the plant cell host such that it is transfected, and will, under appropriate conditions, express the DNA encoding Apo-3 or Apo-2LI. In addition, regulatory and signal sequences compatible with plant cells are available, such as the nopaline synthase promoter and polyadenylation signal sequences [Depicker et al., J. Mol. Appl. Gen., 1:561 (1982)]. In addition, DNA segments isolated from the upstream region of the T-DNA 780 gene are capable of activating or increasing transcription levels of plant-expressible genes in recombinant DNA-containing plant tissue [EP 321,196 published Jun. 21, 1989].

Propagation of vertebrate cells in culture (tissue culture) is also well known in the art [See, e.g., Tissue Culture, Academic Press, Kruse and Patterson, editors (1973)]. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; and FS4 cells.

Host cells are transfected and preferably transformed with the above-described expression or cloning vectors and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published Jun. 29, 1989. In addition, plants may be transfected using ultrasound treatment as described in WO 91/00358 published Jan. 10, 1991.

For mammalian cells without such cell walls, the calcium phosphate precipitation method of Graham and van der Eb, Virology, 52:456-457 (1973) is preferred. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

4. Culturing the Host Cells

Prokaryotic cells used to produce Apo-3 or Apo-2LI may be cultured in suitable media as described generally in Sambrook et al., supra.

The mammalian host cells may be cultured in a variety of media. Examples of commercially available media include Ham's F10 (Sigma), Minimal Essential Medium (“MEM”, Sigma), RPMI-1640 (Sigma), and Dulbecco's Modified Eagle's Medium (“DMEM”, Sigma). Any such media may be supplemented as necessary with hormones and/or other growth factors (such as insulin, transferrin, or 25 epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In general, principles, protocols, and practical techniques for maximizing the productivity of mammalian cell cultures can be found in Mammalian Cell Biotechnology: a Practical Approach, M. Butler, ed. (IRL Press, 1991).

The host cells referred to in this disclosure encompass cells in culture as well as cells that are within a host animal.

5. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Various labels may be employed, most commonly radioisotopes, and particularly ³²P. However, other techniques may also be employed, such as using biotin-modified nucleotides for introduction into a polynucleotide. The biotin then serves as the site for binding to avidin or antibodies, which may be labeled with a wide variety of labels, such as radionucleotides, fluorescers or enzymes. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, or luminescent labels.

Antibodies useful for immunohistochemical staining and/or assay of sample fluids may be either monoclonal or polyclonal, and may be prepared in any mammal. Conveniently, the antibodies may be prepared against a native sequence Apo-3 polypeptide or Apo-2LI polypeptide or against a synthetic peptide based on the DNA sequences provided herein or against exogenous sequence fused to Apo-3 or Apo-2LI DNA and encoding a specific antibody epitope.

6. Purification of Polypeptide

Apo-2LI preferably is recovered from the culture medium as a secreted polypeptide, although it also may be recovered from host cell lysates when directly produced without a secretory signal. Forms of Apo-3 may be recovered from culture medium or from host cell lysates. If the Apo-3 or Apo-2LI is membrane-bound, it can be released from the membrane using a suitable detergent solution (e.g. Triton-X 100) or its extracellular region may be released by enzymatic cleavage.

When Apo-3 or Apo-2LI is produced in a recombinant cell other than one of human origin, the polypeptide is free of proteins or polypeptides of human origin. However, it may be desired to purify the polypeptide from recombinant cell proteins or polypeptides to obtain preparations that are substantially homogeneous as to Apo-3 or Apo-2LI. As a first step, the culture medium or lysate may be centrifuged to remove particulate cell debris. Apo-3 or Apo-2LI thereafter is purified from contaminant soluble proteins and polypeptides, with the following procedures being exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; and protein A Sepharose columns to remove contaminants such as IgG.

Apo-3 or Apo-2LI variants in which residues have been deleted, inserted, or substituted can be recovered in the same fashion as native sequence polypeptides, taking account of any substantial changes in properties occasioned by the variation. For example, preparation of an Apo-3 fusion with another protein or polypeptide, e.g., a bacterial or viral antigen, immunoglobulin sequence, or receptor sequence, may facilitate purification; an immunoaffinity column containing antibody to the sequence can be used to adsorb the fusion polypeptide. Other types of affinity matrices also can be used.

A protease inhibitor such as phenyl methyl sulfonyl fluoride (PMSF) also may be useful to inhibit proteolytic degradation during purification, and antibiotics may be included to prevent the growth of adventitious contaminants. One skilled in the art will appreciate that purification methods suitable for the native sequence polypeptide may require modification to account for changes in the character of the polypeptide or its variants upon expression in recombinant cell culture.

7. Covalent Modifications of Polypeptides

Covalent modifications of Apo-3 or Apo-2LI are included within the scope of this invention. One type of covalent modification of these polypeptides is introduced into the molecule by reacting targeted amino acid residues with an organic derivatizing agent that is capable of reacting with selected side chains or the N- or C- terminal residues.

Derivatization with bifunctional agents is useful for crosslinking the polypeptide to a water-insoluble support matrix or surface for use in the method for purifying anti-Apo-3 or anti-Apo-2LI antibodies, and vice-versa. Derivatization with one or more bifunctional agents will also be useful for crosslinking, for instance, Apo-3 molecules to generate Apo-3 dimers. Such dimers may increase binding avidity and extend half-life of the molecule in vivo. Commonly used crosslinking agents include, e.g., 1,1-bis(diazoacetyl)-2-phenylethane, glutaraldehyde, N-hydroxy-succinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group. The modified forms of the residues fall within the scope of the present invention.

Another type of covalent modification included within the scope of this invention comprises altering the native glycosylation pattern of the polypeptide. “Altering the native glycosylation pattern” is intended for purposes herein to mean deleting one or more carbohydrate moieties found in native sequence Apo-3 or Apo-2LI, and/or adding one or more glycosylation sites that are not present in the native sequence Apo-3 or Apo-2LI, respectively.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxylamino acid, most commonly serine or threonine, although 5-hydroxyproline or 5-hydroxylysine may also be used.

Addition of glycosylation sites may be accomplished by altering the amino acid sequence such that it contains one or more of the above-described tripeptide sequences (for N-linked glycosylation sites). The alteration may also be made by the addition of, or substitution by, one or more serine or threonine residues to the native sequence Apo-3 or Apo-2LI (for O-linked glycosylation sites). The amino acid sequence may optionally be altered through changes at the DNA level, particularly by mutating the DNA encoding the Apo-3 or Apo-2LI polypeptide at preselected bases such that codons are generated that will translate into the desired amino acids. The DNA mutation(s) may be made using methods described above and in U.S. Pat. No. 5,364,934, supra.

Another means of increasing the number of carbohydrate moieties on the polypeptide is by chemical or enzymatic coupling of glycosides to the polypeptide. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published Sep. 11, 1987, and in Aplin and Wriston, CRC Crit. Rev. Biochem., pp. 259-306 (1981).

Removal of carbohydrate moieties present on the polypeptide may be accomplished chemically or enzymatically or by mutational substitution of codons encoding for amino acid residues that serve as targets for glycosylation. For instance, chemical deglycosylation by exposing the polypeptide to the compound trifluoromethanesulfonic acid, or an equivalent compound can result in the cleavage of most or all sugars except the linking sugar (N-acetylglucosamine or N-acetylgalactosamine), while leaving the polypeptide intact. Chemical deglycosylation is described by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Glycosylation at potential glycosylation sites may be prevented by the use of the compound tunicamycin as described by Duksin et al., J. Biol. Chem., 257:3105-3109 (1982). Tunicamycin blocks the formation of protein-N-glycoside linkages.

Another type of covalent modification comprises linking the Apo-3 or Apo-2LI polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

8. Chimeric Molecules

The present invention also provides chimeric molecules comprising Apo-3 or Apo-2LI fused to another, heterologous polypeptide or amino acid sequence.

In one embodiment, the chimeric molecule comprises a fusion of the Apo-3 or Apo-2LI with a tag polypeptide which provides an epitope to which an anti-tag antibody can selectively bind. The epitope tag is generally placed at the amino- or carboxyl- terminus of the Apo-3 or Apo-2LI, respectively. The presence of such epitope-tagged forms can be detected using an antibody against the tag polypeptide. Also, provision of the epitope tag enables the Apo-3 or Apo-2LI to be readily purified by affinity purification using an anti-tag antibody or another type of affinity matrix that binds to the epitope tag.

Various tag polypeptides and their respective antibodies are well known in the art. Examples include the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineerinq, 3(6) :547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol.

Chem., 266:14163-14166 (1991)]; and the T7 gene 10 protein peptide tag [Lutz-Freyermuth et al., Proc. Natl. Acad. Sci. USA, 87:6393-6397 (1990)]. Once the tag polypeptide has been selected, an antibody thereto can be generated using the techniques disclosed herein.

Generally, epitope-tagged- Apo-3 or Apo-2LI may be constructed and produced according to the methods described above. Apo-3 or Apo-2LI-tag polypeptide fusions are preferably constructed by fusing the cDNA sequence encoding the Apo-3 or Apo-2LI portion in-frame to the tag polypeptide DNA sequence and expressing the resultant DNA fusion construct in appropriate host cells. Ordinarily, when preparing the tag polypeptide chimeras of the present invention, nucleic acid encoding the Apo-3 or Apo-2LI will be fused at its 3′ end to nucleic acid encoding the N-terminus of the tag polypeptide, however 5′ fusions are also possible. For example, a polyhistidine sequence of about 5 to about 10 histidine residues may be fused at the N- terminus or the C- terminus and used as a purification handle in affinity chromatography.

Epitope-tagged Apo-3 or Apo-2LI can be purified by affinity chromatography using the anti-tag antibody. The matrix to which the affinity antibody is attached may include, for instance, agarose, controlled pore glass or poly(styrenedivinyl)benzene. The epitope-tagged polypeptide can then be eluted from the affinity column using techniques known in the art.

In another embodiment, the chimeric molecule comprises an Apo-3 or Apo-2LI polypeptide fused to an immunoglobulin sequence or other heterologous sequence. Optionally, the chimeric molecule comprises an Apo-2LI fused to an immunoglobulin constant domain or TNFR sequence. The chimeric molecule may also comprise a particular domain sequence of Apo-3, such as the extracellular domain sequence of native Apo-3 fused to an immunoglobulin sequence. This includes chimeras in monomeric, homo- or heteromultimeric, and particularly homo- or heterodimeric, or - tetrameric forms; optionally, the chimeras may be in dimeric forms or homodimeric heavy chain forms. Generally, these assembled immunoglobulins will have known unit structures as represented by the following diagrams.

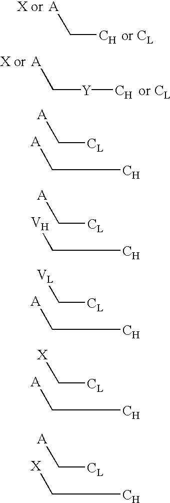

A basic four chain structural unit is the form in which IgG, IgD, and IgE exist. A four chain unit is repeated in the higher molecular weight immunoglobulins; IgM generally exists as a pentamer of basic four-chain units held together by disulfide bonds. IgA globulin, and occasionally IgG globulin, may also exist in a multimeric form in serum. In the case of multimers, each four chain unit may be the same or different.

The following diagrams depict some exemplary monomer, homo- and heterodimer and homo- and heteromultimer structures. These diagrams are merely illustrative, and the chains of the multimers are believed to be disulfide bonded in the same fashion as native immunoglobulins.

| | |
|---|---|
| monomer: | 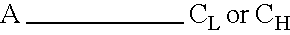 |
| homodimer: | 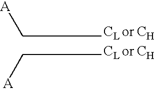 |
| heterodimer: | 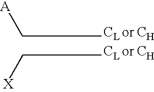 |
| homotetramer: | 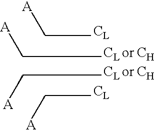 |
| heterotetramer: | 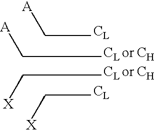 |
| and | 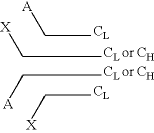 |

In the foregoing diagrams, “A” means an Apo-3 sequence, Apo-2LI sequence, or an Apo-3 or Apo-2LI sequence fused to a heterologous sequence; X is an additional agent, which may be the same as A or different, a portion of an immunoglobulin superfamily member such as a variable region or a variable region-like domain, including a native or chimeric immunoglobulin variable region, a toxin such a pseudomonas exotoxin or ricin, or a sequence functionally binding to another protein, such as other cytokines (i.e., IL-1, interferon-y) or cell surface molecules (i.e., NGFR, CD40, OX40, Fas antigen, T2 proteins of Shope and myxoma poxviruses), or a polypeptide therapeutic agent not otherwise normally associated with a constant domain; Y is a linker or another receptor sequence; and V_{L}, V_{H}, C_{L} and C_{H} represent light or heavy chain variable or constant domains of an immunoglobulin. tructures comprising at least one CRD of an Apo-3 or Apo-2LI sequence as “AA” and another cell-surface protein having a repetitive pattern of CRDs (such as TNFR) as “X” are specifically included.

It will be understood that the above diagrams are merely exemplary of the possible structures of the chimeras of the present invention, and do not encompass all possibilities. For example, there might desirably be several different “A”s, “X”s, or “Y”s in any of these constructs. Also, the heavy or light chain constant domains may be originated from the same or different immunoglobulins. All possible permutations of the illustrated and similar structures are all within the scope of the invention herein.

In general, the chimeric molecules can be constructed in a fashion similar to chimeric antibodies in which a variable domain from an antibody of one species is substituted for the variable domain of another species. See, for example, EP 0 125 023; EP 173,494; Munro, Nature, 312:597 (13 December 1984); Neuberger et al., Nature, 312:604-608 (Dec. 13, 1984); Sharon et al., Nature, 309:364-367 (May 24, 1984); Morrison et al., Proc. Nat'l. Acad. Sci.

USA, 81:6851-6855 (1984); Morrison et al., Science, 229:1202-1207 (1985); Boulianne et al., Nature, 312:643-646 (Dec. 13, 1984); Capon et al., Nature, 337:525-531 (1989); Traunecker et al., Nature, 339:68-70 (1989).

Alternatively, the chimeric molecules may be constructed as follows. The DNA including a region encoding the desired sequence, such as an Apo-3, Apo-2LI and/or TNFR sequence, is cleaved by a restriction enzyme at or proximal to the 3′ end of the DNA encoding the immunoglobulin-like domain(s) and at a point at or near the DNA encoding the N-terminal end of the Apo-3, Apo-2LI, or TNFR polypeptide (where use of a different leader is contemplated) or at or proximal to the N-terminal coding region for TNFR (where the native signal is employed). This DNA fragment then is readily inserted proximal to DNA encoding an immunoglobulin light or heavy chain constant region and, if necessary, the resulting construct tailored by deletional mutagenesis. Preferably, the Ig is a human immunoglobulin when the chimeric molecule is intended for in vivo therapy for humans. DNA encoding immunoglobulin light or heavy chain constant regions is known or readily available from cDNA libraries or is synthesized. See for example, Adams et al., Biochemistry, 19:2711-2719 (1980); Gough et al., Biochemistry, 19:2702-2710 (1980); Dolby et al., Proc. Natl. Acad. Sci., USA, 77:6027-6031 (1980); Rice et al., Proc. Natl. Acad. Sci., 79:7862-7865 (1982); Falkner et al., Nature, 298:286-288 (1982); and Morrison et al., Ann. Rev. Immunol., 2:239-256 (1984).

Further details of how to prepare such fusions are found in publications concerning the preparation of immunoadhesins. Immunoadhesins in general, and CD4-Ig fusion molecules specifically are disclosed in WO 89/02922, published Apr. 6, 1989). Molecules comprising the extracellular portion of CD4, the receptor for human immunodeficiency virus (HIV), linked to IgG heavy chain constant region are known in the art and have been found to have a markedly longer half-life and lower clearance than the soluble extracellular portion of CD4 [Capon et al., supra; Byrn et al., i Nature, 344:667 (1990)]. The construction of specific chimeric TNFR-IgG molecules is also described in Ashkenazi et al. Proc. Natl. Acad. Sci., 88:10535-10539 (1991); Lesslauer et al. [J. Cell. Biochem. Supplement 15F, 1991, p. 115 (P 432)]; and Peppel and Beutler, J.

Cell. Biochem. Supplement 15F, 1991, p. 118 (P 439)].

B. Therapeutic and Non-therapeutic Uses for Apo-3 and Apo-2LI

Apo-3, as disclosed in the present specification, can be employed therapeutically to induce apoptosis or NF-KB or JNK mediated gene expression in mammalian cells. This therapy can be accomplished for instance, using in vivo or ex vivo gene therapy techniques and includes the use of the death domain sequences disclosed herein. The Apo-3 chimeric molecules (including the chimeric molecules containing the extracellular domain sequence of Apo-3) comprising immunoglobulin sequences can also be employed therapeutically to inhibit apoptosis or NF-KB induction or JNK activation. Apo-2LI, as disclosed in the application, can be employed therapeutically to inhibit mammalian cell apoptosis in vivo or ex vivo. Generally, the methods comprise exposing the cells to an effective amount of the Apo-2LI.

The Apo-3 and Apo-2LI of the invention also have utility in non-therapeutic applications. Nucleic acid sequences encoding the Apo-3 or Apo-2LI may be used as a diagnostic for tissue-specific typing. For example, procedures like in situ hybridization, Northern and Southern blotting, and PCR analysis may be used to determine whether DNA and/or RNA encoding the polypeptide is present in the cell type(s) being evaluated. Apo-3 or Apo-2LI nucleic acid will also be useful for the preparation of Apo-3 or Apo-2LI, respectively by the recombinant techniques described herein.

The isolated Apo-3 or Apo-2LI may be used in quantitative diagnostic assays as a control against which samples containing unknown quantities of Apo-3 or Apo-2LI may be prepared. Apo-3 preparations are also useful in generating antibodies, as standards in assays for Apo-3 or Apo-2LI (e.g., by labeling Apo-3 or Apo-2LI for use as a standard in a radioimmunoassay, radioreceptor assay, or enzyme-linked immunoassay), in affinity purification techniques, and in competitive-type receptor binding assays when labeled with, for instance, radioiodine, enzymes, or fluorophores.

Modified forms of the Apo-3, such as the Apo-3-IgG chimeric molecules (immunoadhesins) described above, can be used as immunogens in producing anti-Apo-3 antibodies.

Nucleic acids which encode Apo-3 or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding Apo-3 or an appropriate sequence thereof can be used to clone genomic DNA encoding Apo-3 in accordance with established techniques and the genomic sequences used to generate transgenic animals that contain cells which express DNA encoding Apo-3. Methods for generating transgenic animals, particularly animals such as mice or rats, have become conventional in the art and are described, for example, in U.S. Pat. Nos. 4,736,866 and 4,870,009. Typically, particular cells would be targeted for Apo-3 transgene incorporation with tissue-specific enhancers. Transgenic animals that include a copy of a transgene encoding Apo-3 introduced into the germ line of the animal at an embryonic stage can be used to examine the effect of increased expression of DNA encoding Apo-3. Such animals can be used as tester animals for reagents thought to confer protection from, for example, pathological conditions associated with excessive apoptosis. In accordance with this facet of the invention, an animal is treated with the reagent and a reduced incidence of the pathological condition, compared to untreated animals bearing the transgene, would indicate a potential therapeutic intervention for the pathological condition. In another embodiment, transgenic animals that carry a soluble form of Apo-3 such as the Apo-3 ECD or an immunoglobulin chimera of such form could be constructed to test the effect of chronic neutralization of the ligand of Apo-3.

Alternatively, non-human homologues of Apo-3 can be used to construct a Apo-3 “knock out” animal which has a defective or altered gene encoding Apo-3 as a result of homologous recombination between the endogenous gene encoding Apo-3 and altered genomic DNA encoding Apo-3 introduced into an embryonic cell of the animal. For example, cDNA encoding Apo-3 can be used to clone genomic DNA encoding Apo-3 in accordance with established techniques. A portion of the genomic DNA encoding Apo-3 can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-151]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the Apo-3 polypeptide, including for example, development of tumors.

C. Antibody Preparation

The present invention further provides anti-Apo-3 antibodies and anti-Apo-2LI antibodies. Such antibodies may be prepared as follows. Exemplary antibodies include polyclonal, monoclonal, humanized, bispecific, and heteroconjugate antibodies.

1. Polyclonal Antibodies

The antibodies may comprise polyclonal antibodies.

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the Apo-3 or Apo-2LI polypeptide or a fusion protein thereof. An example of a suitable immunizing agent is a Apo-3-IgG fusion protein or chimeric molecule (including an Apo-3 ECD-IgG fusion protein). Cells expressing Apo-3 at their surface may also be employed. It may be useful to conjugate the immunizing agent to a protein known to be immunogenic in the mammal being immunized. Examples of such immunogenic proteins which may be employed include but are not limited to keyhole limpet hemocyanin, serum albumin, bovine thyroglobulin, and soybean trypsin inhibitor. An aggregating agent such as alum may also be employed to enhance the mammal's immune response. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation. The mammal can then be bled, and the serum assayed for antibody titer. If desired, the mammal can be boosted until the antibody titer increases or plateaus.

2. Monoclonal Antibodies

The antibodies may, alternatively, be monoclonal antibodies. Monoclonal antibodies may be prepared using hybridoma methods, such as those described by Kohler and Milstein, supra. In a hybridoma method, a mouse, hamster, or other appropriate host animal, is typically immunized (such as described above) with an immunizing agent to elicit lymphocytes that produce or are capable of producing antibodies that will specifically bind to the immunizing agent. Alternatively, the lymphocytes may be immunized in vi tro.

The immunizing agent will typically include the Apo-3 or Apo-2LI polypeptide or a fusion protein thereof. An example of a suitable immunizing agent is a Apo-3-IgG fusion protein or chimeric molecule. Cells expressing Apo-3 or Apo-2LI at their surface may also be employed. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against Apo-3 or Apo-2LI. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields an F(ab′)₂ fragment that has two antigen combining sites and is still capable of cross-linking antigen.

The Fab fragments produced in the antibody digestion also contain the constant domains of the light chain and the first constant domain (CH₁) of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

3. Humanized Antibodies

The antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-327 (1988); and Presta, Curr. Op. Struct. Biol., 2:593-596 (1992)].

Methods for humanizing non-human antibodies are well known in the art. Generally, a humanized antibody has one or more 35 amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody [Sims et al., J. Immunol. 151:2296 (1993); Chothia and Lesk, J. Mol. Biol., 196:901 (1987)]. Another method uses a particular framework derived from the consensus sequence of all human antibodies of a particular subgroup of light or heavy chains. The same framework may be used for several different humanized antibodies [Carter et al., Proc. Natl. Acad. Sci. USA, 89:4285 (1992); Presta et al., J. Immunol., 151:2623 (1993)].

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding [see, WO 94/04679 published Mar. 3, 1994].

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge [see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)]. Human antibodies can also be produced in phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)].

4. Bispecific Antibodies

Bispecific antibodies are monoclonal, preferably human or humanized, antibodies that have binding specificities for at least two different antigens. In the present case, one of the binding specificities is for the Apo-3 or the Apo-2LI, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

According to a different and more preferred approach, antibody variable domains with the desired binding specificities (antibody-antigen combining sites) are fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy-chain/light-chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details of generating bispecific antibodies see, for example, Suresh et al., Methods in Enzymology, 121:210 (1986).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/20373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

D. Antibody Therapeutic and Non-therapeutic Uses

The antibodies of the invention have therapeutic utility. Agonistic Apo-3 antibodies, for instance, may be employed to activate or stimulate apoptosis in cancer cells. Alternatively, antagonistic antibodies may be used to block excessive apoptosis (for instance in neurodegenerative disease) or to block potential autoimmune/inflammatory effects of Apo-3 resulting from NF-KB activation and/or JNK activation.

The antibodies may further be used in diagnostic assays. For example, Apo-3 antibodies or Apo-2LI antibodies may be used in diagnostic assays for Apo-3 or Apo-2LI, respectively, e.g., detecting its expression in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Apo-3 or Apo-2LI antibodies also are useful for the affinity purification of Apo-3 or Apo-2LI from recombinant cell culture or natural sources. In this process, the antibodies are immobilized on a suitable support, such a Sephadex resin or filter paper, using methods well known in the art. The immobilized antibody then is contacted with a sample containing the Apo-3 or Apo-2LI to be purified, and thereafter the support is washed with a suitable solvent that will remove substantially all the material in the sample except the Apo-3 or Apo-2LI, which is bound to the immobilized antibody. Finally, the support is washed with another suitable solvent that will release the Apo-3 or Apo-2LI from the antibody.

E. Kits and Articles of Manufacture

In a further embodiment of the invention, there are provided articles of manufacture and kits containing Apo-3, Apo-2LI, or Apo-3 or Apo-2LI antibodies which can be used, for instance, for the therapeutic or non-therapeutic applications described above. The article of manufacture comprises a container with a label. Suitable containers include, for example, bottles, vials, and test tubes. The containers may be formed from a variety of materials such as glass or plastic. The container holds a composition which includes an active agent that is effective for therapeutic or non-therapeutic applications, such as described above. The active agent in the composition is Apo-3 or Apo-2LI, or an Apo-3 or Apo-2LI antibody. The label on the container indicates that the composition is used for a specific therapy or non-therapeutic application, and may also indicate directions for either in vivo or in vitro use, such as those described above.

The kit of the invention will typically comprise the container described above and one or more other containers comprising materials desirable from a commercial and user standpoint, including buffers, diluents, filters, needles, syringes, and package inserts with instructions for use.

The following examples are offered for illustrative purposes only, and are not intended to limit the scope of the present invention in any way.

All references cited in the present specification are hereby incorporated by reference in their entirety.

### EXAMPLES

All restriction enzymes referred to in the examples were purchased from New England Biolabs and used according to manufacturer's instructions. All other commercially available reagents referred to in the examples were used according to manufacturer's instructions unless otherwise indicated. The source of those cells identified in the following examples, and throughout the specification, by ATCC accession numbers is the American Type Culture Collection, Rockville, Md.

### EXAMPLE 1

### Isolation of CDNA clones Encoding Human Apo-2LI

To isolate a cDNA for Apo-2LI, a lambda gt10 bacteriophage library of human thymus cDNA (about 1×106 clones) (HL1074a, commercially available from Clontech) was screened by hybridization with synthetic oligonucleotide probes based on an EST sequence (GenBank locus H41522), which showed some degree of homology to human Fas/Apo-1. The EST sequence of H41522 is 433 bp and when translated in its +1 frame, shows 20 identities to a 78 amino acid region of human Fas/Apo-1. The sequence of H41522 is as follows:

| | |
|---|---|
| CTGCTGGGGGCCCGGGCCAGNGGCGGCACTCGTAGCCCCAGGTGTGACTGTGCCGGTGAC | (SEQ ID NO:2) |
| TTCCACAAGAAGATTGGTCTGTTTTGTTGCAGAGGCTGCCCAGCGGGGCAACTACCTGAA | |
| GGCCCCTTGCACGGAGCCCTGCGCAACTCCACCTGCCTTGTGTGTCCCCAAGACACCTTC | |
| TTGGCCTGGGAGAACCACCATAATTCTGAATGTGCCCGCTGCCAGGCCTGTGATGAGCAG | |
| GCCTCCCAGGTGGCGCTGGAGAACTGTTCAGCAGTGGCCGACACCCGCTGTGGCTGTAAG | |
| CAGGGCTGGTTTGTGGAGTGCCAGGGTCAGCCAATGTGTCAGCAGTTTCACCCTTCTAAT | |
| GCCAACCATGCCTAGACTGCGGGGCCCTGCAACGCAACACACGGCTAATNTGTTTCCCGC | |
| AGAGATNATTGTT | |

The oligonucleotide probes employed in the screening were 28 bp long, with the following respective sequences:

CCCGCTGCCAGGCCTGTGATGAGCAGGC (SEQ ID NO:3)

CAGGGCCCCGCAGTCTAGGCATGGTTGG (SEQ ID NO:4)

Hybridization was conducted with a 1:1 mixture of the two probes overnight at room temperature in buffer containing 20% formamide, 5X SSC, 10% dextran sulfate, 0.1% NaPIPO₄, 0.05 M NaPO₄, 0.05 mg salmon sperm DNA, and 0.1% sodium dodecyl sulfate, followed consecutively by one wash at room temperature in 6X SSC, two washes at 37° C. in 1X SSC/0.1% SDS, two washes at 37° C. in 0.5X SSC/0.1% SDS, and two washes at 37° C. in 0.2X SSC/O.1% SDS. Four positive clones were identified in the cDNA library, and the positive clones were confirmed to be specific by PCR using the above hybridization probes as PCR primers. Single phage plaques containing each of the four positive clones were isolated by limiting dilution and the DNA was purified using a Wizard Lambda Prep DNA purification kit (commercially available from Promega).

The CDNA inserts from the four bacteriophage clones were excised from the vector arms by digestion with EcoRI, gel-purified, and subcloned into pRK7 [EP 278,776 published August 17, 1988] that was predigested with EcoRI. Three of the clones (18.1, 24.1, and 28.1) contained an identical open reading frame; therefore further analysis was done with only one clone, 18.1. Clone 18.1 was approximately 1.4 kb long.

The entire nucleotide sequence of Apo-2LI is shown in FIG. 1 (SEQ ID NO:5). The cDNA contained one open reading frame with a translational initiation site assigned to the ATG codon at nucleotide positions 377-379. The surrounding sequence at this site is in reasonable agreement with the proposed consensus sequence for initiation sites [Kozak, J. Cell. Biol., 115:887-903 (1991)]. The open reading frame ends at the termination codon TAA at nucleotide positions 919-921.

The predicted amino acid sequence of the Apo-2LI encoded by clone 18.1 contains 181 amino acids, and has a calculated molecular weight of approximately 19.3 kDa and an isoelectric point of approximately 7.1. Hydropathy analysis indicated the presence of a hydrophobic signal sequence at the N-terminus of approximately amino acids. Two potential N-linked glycosylation sites are located at residues 67 and 105 of the polypeptide precursor.

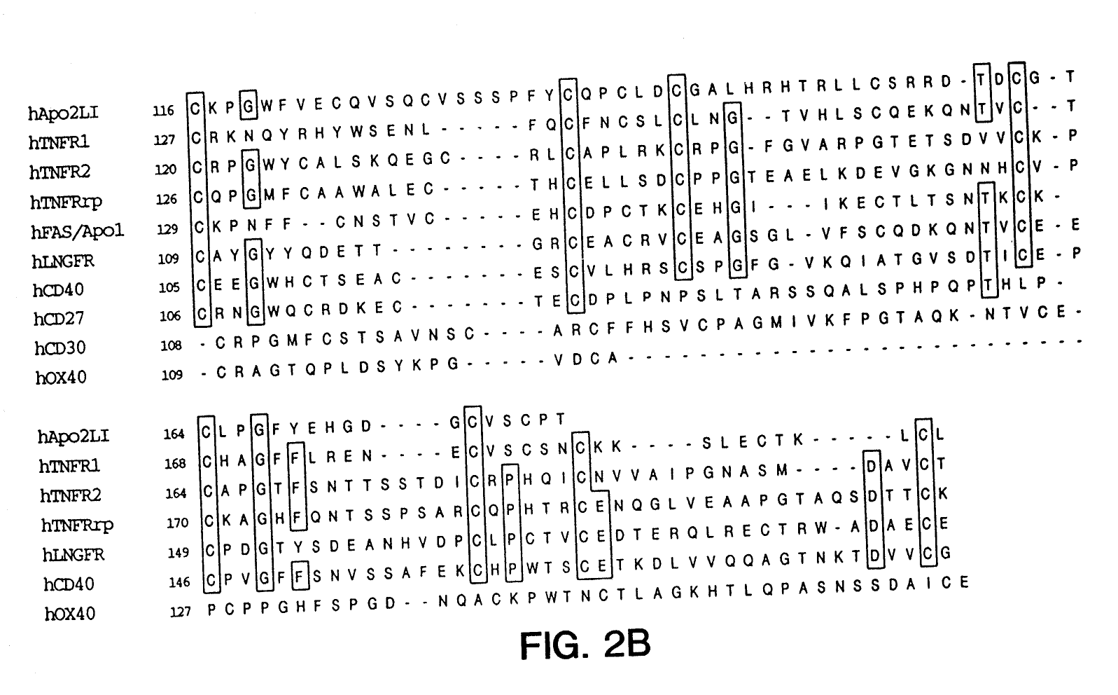

An alignment (using the Align™ computer program) of the amino acid sequence encoded by clone 18.1 with the extracellular regions of other known members of the human TNF receptor family showed the following percentages of identity: 30.2% identity to Fas/Apo-1; 28.7% to type 1 TNF receptor (TNFR1); 22.5% to the low affinity NGF receptor (LNGFR) and to CD40; 21.8% to CD30; 21.5% to CD27; 21.4% to OX40; 20.5% to type 2 TNF receptor (TNFR2); 20.1% to TNF receptor related protein (TNFRrp). (See also, FIG. 2)

TNF receptor family proteins are typically characterized by the presence of multiple (usually four) cysteine-rich domains in their extracellular regions—each cysteine-rich domain being approximately 45 amino acid long and contains approximately 6, regularly spaced, cysteine residues. Based on the crystal structure of the type 1 TNF receptor, the cysteines in each domain typically form three disulfide bonds in which usually cysteines 1 and 2, 3 and 5, and 4 and 6 are paired together. Applicants found that the polypeptide encoded by clone 18.1 contains three cysteine-rich domains and an apparently truncated fourth cysteine-rich domain that contains only three cysteines and stops 5 amino acids C-terminally to the third cysteine.

Amino acids 1 to 181 of the Apo-2LI clone 18.1 shown in FIG. 1 (SEQ ID NO:l) are identical to amino acids 1 to 181 of the Apo-3 polypeptide, as described in Example 4 below, and shown in FIG. 4 (SEQ ID NO:6). Compared to Apo-3 polypeptide described in Example 4 below, the polypeptide encoded by clone 18.1 is truncated within the C-terminal region of the ECD and lacks some extracellular sequence as well as the transmembrane and cytoplasmic sequences of Apo-3. The truncation is believed to occur by alternative splicing of the mRNA which introduces a stop codon 5 amino acids downstream of the third cysteine of the fourth cysteine-rich domain. The 3′ untranslated region is distinct from that of the Apo-3 clone FL8A.53 and contains a distinct polyadenylation site, suggesting that clone 18.1 represents a naturally-occurring mRNA.

### EXAMPLE 2

### Expression of Apo-2LI Clone 18.1

A pRK7 plasmid (described in Example 1) containing the Apo-2LI cDNA (as described in Example 1) in the forward orientation, or a control pRK5 plasmid [Schall et al., Cell, 61:361-370 (1990); Suva, Science, 237:893-896 (1987)] containing the Apo-2LI cDNA in the reverse orientation, were transfected transiently into human 293 cells (ATCC CRL 1573) by calcium phosphate precipitation. After 24 hours, the medium was replaced by serum free medium, and the cells were incubated for an additional 48 hours. The serum free conditioned media were then collected, cleared by centrifugation, and concentrated 5-fold by centrifugation in centricon tubes.

### EXAMPLE 3

### Expression of Apo-2LI Immunoadhesin

An immunoadhesin was constructed that consisted of the Apo-2LI coding region (as described in Example 1), including its endogenous signal sequence, fused C-terminally to residues 183-211 of type 1 TNF receptor, which was fused in turn to the hinge and Fc regions of human IgG1 heavy chain, as described previously by Ashkenazi et al., supra.

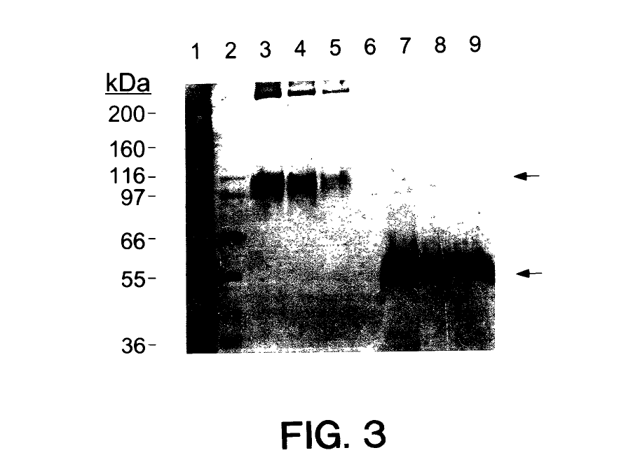

The pRK5 plasmid encoding the chimeric Apo-2LI immunoadhesin was transiently transfected into human 293 cells (described in Example 2) by calcium phosphate precipitation. After 24 hours, the medium was replaced by serum free medium, and the cells were incubated for an additional 6 days. The serum free conditioned media were then collected, cleared by centrifugation, and purified by protein A affinity chromatography, as described previously by Ashkenazi et al., supra. Gel electrophoresis showed that the purified protein exhibited a molecular weight of approximately 110 kDa under non-reducing conditions (FIG. 3, lanes 3-5) and approximately 55 kDa under reducing conditions (100 mM DTT, FIG. 3, lanes 7-9), thus indicating a disulfide-bonded homodimeric immunoadhesin structure. Higher molecular weight bands observed for non-reducing conditions are believed to be due to some aggregation of the immunoadhesin during sample preparation.

### EXAMPLE 4

### Isolation of cDNA clones Encoding Human Apo-3

Human fetal heart and human fetal lung lgt10 bacteriophage cDNA libraries (both purchased from Clontech) were screened by hybridization with synthetic oligonucleotide probes based on an EST (Genbank locus W71984), which showed some degree of homology to the intracellular domain (ICD) of human TNFR1 and CD95. W71984 is a 523 bp EST, which in its −1 reading frame has 27 identities to a 43 amino acid long sequence in the ICD of human TNFR1. The oligonucleotide probes used in the screening were 27 and 25 bp long, respectively, with the following sequences: GGCGCTCTGGTGGCCCTTGCAGAAGCC [SEQ ID NO:7] and TTCGGCCGAGAAGTTGAGAAATGTC [SEQ ID NO:8].

Hybridization was done with a 1:1 mixture of the two probes overnight at room temperature in buffer containing 20% formamide, 5X SSC, 10% dextran sulfate, 0.1% NaPiPO₄, 0.05 M NaPO₄, 0.05 mg salmon sperm DNA, and 0.1% sodium dodecyl sulfate (SDS), followed consecutively by one wash at room temperature in 6X SSC, two washes at 37° C. in 1X SSC/0.1% SDS, two washes at 37° C. in 0.5X SSC/0.1% SDS, and two washes at 37° C. in 0.2X SSC/0.1% SDS. One positive clone from each of the fetal heart (FH20A.57) and fetal lung (FL8A.53) libraries were confirmed to be specific by PCR using the respective above hybridization probes as primers. Single phage plaques containing each of the positive clones were isolated by limiting dilution and the DNA was purified using a Wizard lambda prep DNA purification kit (Promega).

The cDNA inserts were excised from the lambda vector arms by digestion with EcoRI, gel-purified, and subcloned into PRKS that was predigested with EcoRI. The clones were then sequenced in entirety.

Clone FH20A.57 (also referred to as Apo 3 clone FH20.57 deposited as ATCC 55820, as indicated below) contains a single open reading frame with an apparent translational initiation site at nucleotide positions 89-91 and ending at the stop codon found at nucleotide positions 1340-1342 (FIG. 4; SEQ ID NO:9) [Kozak et al., supra] . The cDNA clone also contains a polyadenylation sequence at its 3′ end. The predicted polypeptide precursor is 417 amino acids long and has a calculated molecular weight of approximately 45 kDa and a PI of about 6.4. Hydropathy analysis (not shown) suggested the presence of a signal sequence (residues 1-24), followed by an extracellular domain (residues 25-198), a transmembrane domain (residues 199-224), and an intracellular domain (residues 225-417) (FIG. 4; SEQ ID NO:6). There are two potential N-linked glycosylation sites at amino acid positions 67 and 106.

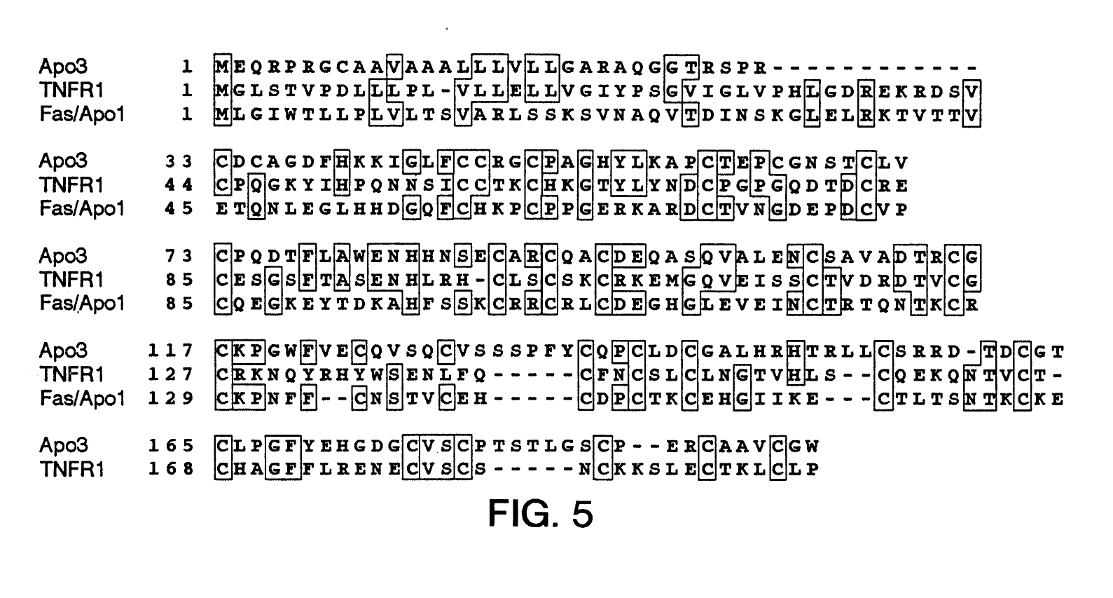

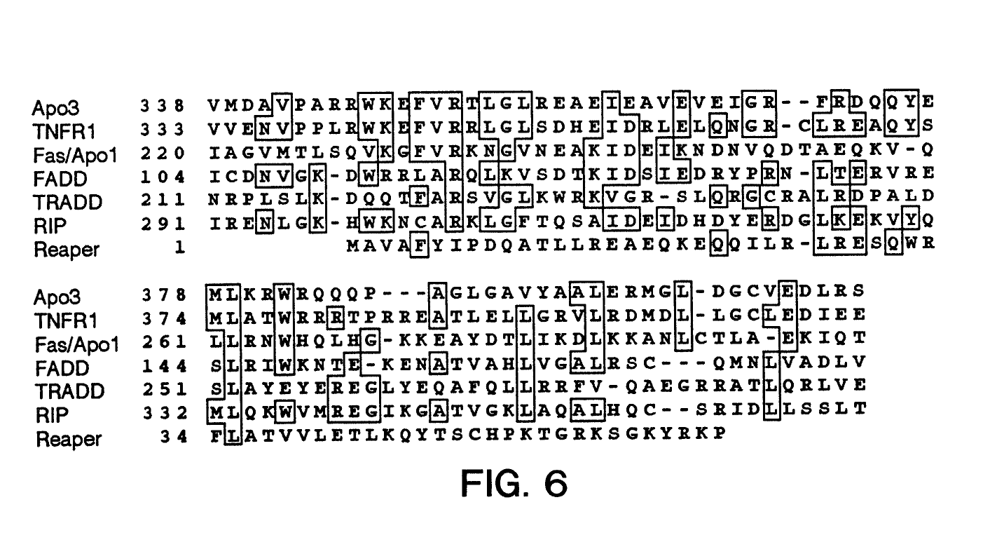

The ECD contains 4 cysteine-rich repeats which resemble the corresponding regions of human TNFRl (4 repeats), of human CD95 (3 repeats) (FIG. 5) and of the other known TNFR family members (not shown). The ICD contains a death domain sequence that resembles the death domains found in the ICD of TNFRl and CD95 and in cytoplasmic death signalling proteins such as human FADD/MORT1, TRADD, RIP, and Drosophila Reaper (FIG. 6). Both globally and in individual regions, Apo-3 is related more closely to TNFR1 than to CD95; the respective amino acid identities are 29.3% and 22.8% overall, 28.2% and 24.7% in the ECD, 31.6% and 18.3% in the ICD, and 47.5% and 20% in the death domain.

The fetal lung cDNA clone, clone 5L8A.53, was identical to the fetal heart clone, with the following two exceptions: (1) it is 172 bp shorter at the 5′ region; and (2) it lacks the Ala residue at position 236, possibly due to differential mRNA splicing via two consecutive splice acceptor consensus sites (FIG. 6).

As mentioned in Example 1 above, amino acids 1 to 181 of the Apo-2LI clone 18.1 shown in FIG. 1 (SEQ ID NO:1) are identical to amino acids 1 to 181 of the Apo-3 polypeptide, shown in FIG. 4 (SEQ ID NO:6).

### EXAMPLE 5

### Expression of Apo-3

A pRK5 mammalian expression plasmid (described in Example 2) carrying clone FH20A.57 (referred to in Example 4) was transfected transiently into HEK293 cells (referred to in the Examples above) by calcium phosphate precipitation and into HeLa-S3 cells (ATCC No. CCL 2.2) by standard electroporation techniques.

Lysates of metabolically labeled transfected 293 cells were analyzed by immunoprecipitation with a mouse antiserum raised against an Apo-2LI-IgG fusion protein. Transfected cells (5×10⁵ per lane) were labeled metabolically by addition of 50 μCi ³⁵S-Met and ³⁵S-Cys to the growth media 24 hours after transfection. After a 6 hour incubation, the cells were washed several times with PBS, lysed and subjected to immunoprecipitation by anti-Apo-3 antiserum as described in Marsters et al., Proc. Natl. Acad. Sci., 92:5401-5405 (1995). The anti-Apo-3 antiserum was raised in mice against a fusion protein containing the Apo-2LI ECD (as described in Example 3).

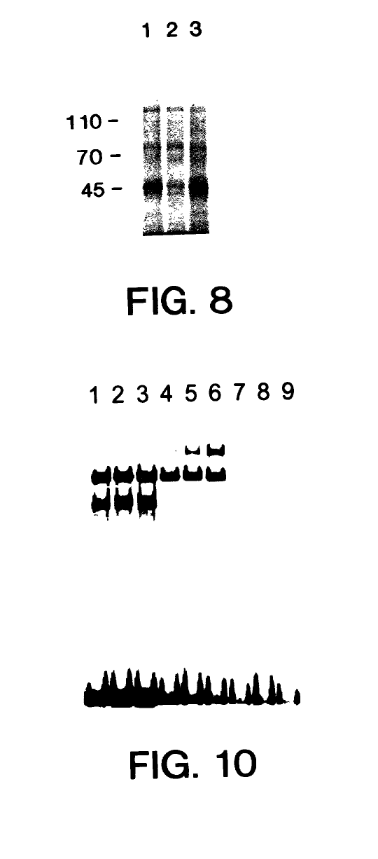

A predominant radioactive band with a relative molecular weight of about 47 kDa was observed in the pRK5-Apo-3-transfected cells, but not in the cells transfected with PRK5 alone (control) (See FIG. 8, lanes 1, 2). Given the potential glycosylation sites of Apo-3, the observed size is consistent with the size of approximately 45 kDa predicted for the Apo-3 polypeptide precursor.

### EXAMPLE 6

### Apoptotic Activity of Apo-3

The transiently transfected HEK293 and HeLa cells described in Example 5 were tested and analyzed for apoptotic activity 36 hours after transfection. Apoptosis was assessed morphologically or quantitated by FACS analysis of cells stained with fluoresceinisothiocyanate (FITC)-conjugated annexin V (Brand Applications) and propidium iodide (PI). The FACS analysis was conducted, using established criteria for apoptotic cell death, namely, the relation of fluorescence staining of the cells with two markers: (a) propidium iodide (PI) which stains the apoptotic cells but not the live cells, and (b) a fluorescent derivative of the protein, annexin V, which binds to the exposed phosphatidylserine found on the surface of apoptotic cells, but not on live cells [Darsynkiewicz et al., Methods in Cell. Biol., 41:15-38 (1994); Fadok et al., J. Immunol., 148:2207-2214 (1992); Koopman et al., Blood, 84:1415-1420 (1994)]. The annexin V-positive/PI negative cells are in early stages of apoptosis and double-positive cells are in late apoptosis, while annexin V-negative/PI-positive cells are necrotic. Apoptosis was also assessed by DNA fragmentation testing.

Microscopic examination of the HEK 293 cells transfected with the pRK5-Apo-3 expression plasmid (see Example 5) showed a substantial loss of cell viability as compared to control cells transfected with pRK5 alone; many of the Apo-3 transfected cells exhibited a characteristic apoptotic morphology of membrane blebbing and loss of cell volume (FIGS. 9 a and b), suggesting cell death by apoptosis [Cohen, Advances in Immunology, 50:55-85 (1990)].

The FACS analysis also revealed that the Apo-3-transfected cells died by apoptosis, by virtue of the presence of exposed phosphatidylserine on their surface (FIGS. 9 e-i). It was found that the transient transfection efficiency of the HEK 293 cells was 60-70%; therefore, to target FACS analysis to cells that had taken up the plasmid DNA, the 293 cells were co-transfected with a pRK5-CD4 expression vector (3 μg) as a marker and gated on CD4-positive cells (using phycoerythrin-conjugated anti-CD4 antibody) for analysis. For the co-transfection, the total amount of plasmid DNA was kept constant, but divided between different plasmids. The Apo-3-transfected cells showed a marked increase in PI and annexin V-FITC staining as compared to pRK5-transfected control cells indicating induction of apoptosis by Apo-3. (FIGS. 9 e and f).

The effect of the dose of plasmid on apoptosis was also tested in the FACS assay. (FIG. 9i). Transfection of 293 cells with either Apo-3 or TNFR1 expression plasmids was associated with a dose-dependent increase in apoptosis; the effect of Apo-3 was more pronounced than that of TNFR1 (FIG. 9i). Similar results were obtained upon Apo-3 transfection of the HeLa cells (data not shown).

Apoptosis was also assayed by extraction of DNA from the cells, terminal transferase-mediated ³²P-labelling of 3′ ends of DNA and 1.5% agarose gel electrophoresis as described by Moore et al., Cytotechnology, 17:1-11 (1995). Analysis of the cellular DNA revealed that the Apo-3-transfected cells showed a marked increase in DNA fragmentation as compared to controls (FIG. 9j, lanes 1, 2). The fragmented DNA migrated on agarose gels as a ladder of bands, indicating internucleosomal DNA cleavage, an indication of programmed cell death [Cohen, supra].

### EXAMPLE 7

### Inhibition Assay Using CrmA

To investigate whether proteases such as ICE and CPP32/Yama play a role in apoptosis-induction by Apo-3, an assay was conducted to determine if CrmA inhibits Apo-3 function.

Co-transfection of HEK293 cells by a pRK5-CrmA expression plasmid (CrmA sequence reported in Ray et al., supra) and pRK5-Apo-3 did not affect the apparent levels of Apo-3 expressed by the cells (FIG. 8, lane 3). CrmA, however, blocked Apo-3 associated apoptosis as analyzed by morphological examination (FIGS. 9 c and d), FACS (FIGS. 9 g and h) and DNA fragmentation (FIG. 9j, lanes 3,4) methods described in Example 6. A similar inhibitory effect of CrmA was observed in Apo-3-transfected HeLa cells (data not shown).

CrmA, a poxvirus-derived inhibitor of the death proteases ICE and CPP32/Yama, blocks death signalling by TNFRl and CD95. Accordingly, the assay results suggest that Apo-3, TNFR1 and CD95 engage a common signalling pathway to activate apoptotic cell death. In particular, the results suggest that proteases such as ICE and CPP32/Yama may be required for Apo-3 induced apoptosis.

### EXAMPLE 8

### Activation of NF-KB by Apo-3

An assay was conducted to determine whether Apo-3 activates NF-KB.

HEK 293 cells were harvested 36 hours after transfection (see Example 5) and nuclear extracts were prepared and 1 μg of nuclear protein was reacted with a ³²P-labelled NF-KB-specific synthetic oligonucleotide probe

ATCAGGGACTTTCCGCTGGGGACTTTCCG (SEQ ID NO:10) [see, also, MacKay et al., J. Immunol., 153:5274-5284 (1994)], alone or together with a 50-fold excess of unlabelled probe, or with an irrelevant ³²P-labelled synthetic oligonucleotide

AGGATGGGAAGTGTGTGATATATCCTTGAT (SEQ ID NO:11). DNA binding was analyzed by an electrophoretic mobility shift assay as described by Hsu et al., supra; Marsters et al., supra, and MacKay et al., supra.

The results are shown in FIG. 10. The radioactive band at the bottom of the gel in all lanes is the free labelled probe, the two other radioactive bands seen in lanes 1-3 represent non-specific interaction, as does the band common to lanes 1-3 and lanes 4-6. The top radioactive band in lanes 4-6 represents the labelled NF-KB probe, whose migration is delayed by specific interaction with activated NF-KB protein in the nuclear extracts.

Apo-3 transfected cells showed a significant increase in NF-KB-specific DNA binding activity relative to pRK5-transfected controls. TNFRl-transfected cells showed NF-KB activation as well; this activation appeared to be enhanced as compared to the Apo-3-transfected cells. The data thus shows that Apo-3 is capable of regulating transcription of inflammatory response genes and in particular, may be linked to a NF-KB activation pathway.

### EXAMPLE 9

### Activation of JNK by Apo-3

An assay was conducted to determine whether Apo-3 activates c-Jun N-terminal kinase (JNK). HEK 293 cells were harvested 36 hours after transfection (see Example 5) and JNK activation was determined by analyzing phosphorylation of c-Jun with a SAPK/JNK assay kit (New England Biolabs) according to manufacturer instructions.

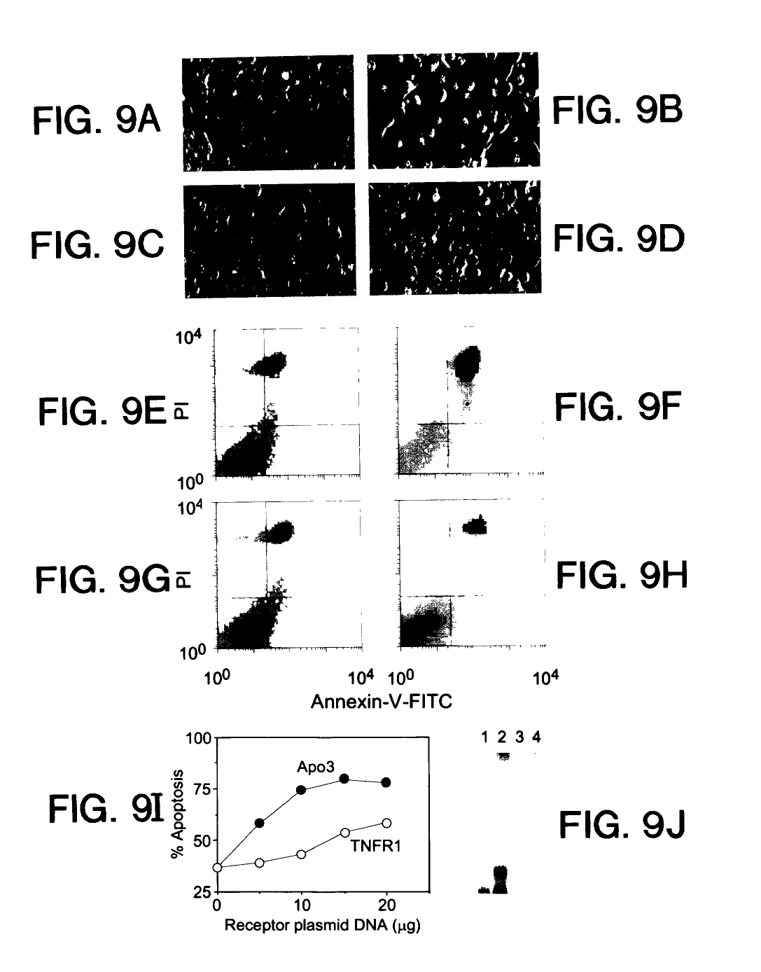

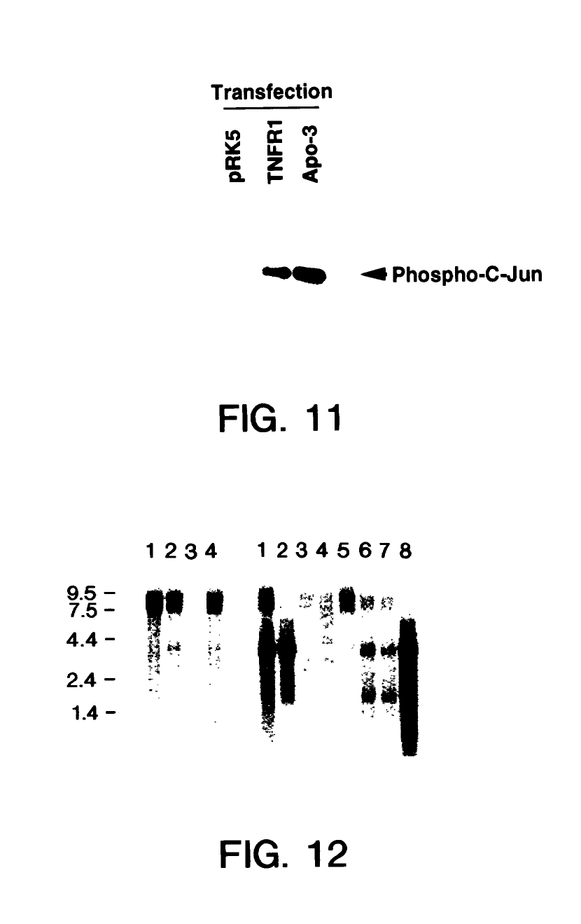

Cell lysates were prepared from HEK 293 cells transfected with 10 μg pRK5, pRK5-TNFR1, or pRK5-Apo-3. JNK was precipitated with a GST-c-Jun fusion protein bound to glutathione-sepharose beads. After washing, the kinase reaction was allowed to proceed in the presence of ATP, and was resolved by SDS-PAGE. Phospho-c-Jun was detected by immunoblot with antibody specific for c-Jun phosphorylated on Ser63, a site important for transcriptional activity, using chemiluminescence detection. The results are shown in FIG. 11.

Apo-3 transfected cells showed a significant level of JNK activation as compared to pRK5 transfected controls. TNFRL transfected cells showed JNK activation as well; this activation appeared to be reduced relative to that seen in Apo-3 transfected cells. The data thus shows that Apo-3 is capable of regulating the stress-response signaling pathway which is also known to be regulated by stimuli such as UV irradiation and various cytokines.

### EXAMPLE 10

### Northern Blot Analysis

Expression of Apo-3 mRNA in human tissues was examined by Northern blot analysis. Human RNA blots were hybridized to a 206 bp ³²P-labelled DNA probe based on the 3′ untranslated region of Apo-3; the probe was generated by PCR with the 27 and 25 bp probes (described in Example 4) as PCR primers. Human fetal RNA blot MTN (Clontech) and human adult RNA blot MTN-II (Clontech) were incubated with the DNA probes. Blots were incubated with the probes in hybridization buffer (5X SSPE; 2X Denhardt's solution; 100 mg/mL denatured sheared salmon sperm DNA; 50% formamide; 2% SDS) for 60 hours at 42° C. The blots were washed several times in 2X SSC; 0.05% SDS for 1 hour at room temperature, followed by a 30 minute wash in 0.1X SSC; 0.1% SDS at 50° C. The blots were developed after overnight exposure.

As shown in FIG. 12, a predominant mRNA transcript of approximately 4 kb was detected in adult spleen, thymus, and peripheral blood lymphocytes, and less abundantly in small intestine, colon, fetal lung, and fetal kidney. Additional transcripts of approximately 7 and 9 kb were seen mainly in fetal brain, lung and kidney, and in adult spleen and ovary. These results suggest that Apo-3 mRNA is expressed in several types of tissues, including both lymphoid and non-lymphoid tissues.

### EXAMPLE 11

### Chromosomal Localization of the Apo-3 gene

Chromosomal localization of the Apo-3 gene was examined by fluorescence in situ hybridization (“FISH”) to normal human lymphocyte chromosomes.

Initial testing by direct hybridization with the Apo-2LI (clone 18.1) cDNA (see Example 1 and FIG. 1) as a probe gave a relatively poor signal to background ratio (data not shown) but suggested that the gene is located on chromosome 1p36. Further testing was conducted using the Apo-3 cDNA probe and FISH mapping [as described by Lichter et al., Science, 247:64-69 (1990)] of a human genomic p1-derived artificial chromosome (PAC) library (obtained from Dr. L. C. Tsui, University of Toronto, Toronto, Canada). The Apo-3 probes were biotinylated and detected with avidin-FITC. The normal human lymphocyte chromosomes were counterstained with PI and DAPI [Heng and Tsui, Chromosome, 102:325-332 (1993)]. In addition to the “direct” FISH using the Apo-3 cDNA as a probe, the probe was used to identify clones in the genomic PAC library that contain the Apo-3 gene, and the PACs were used as confirmatory probes in FISH. The regional assignment of the genomic probe was determined by the analysis of 20 well-spread metaphases.

A positive PAC clone was mapped by FISH to the short arm of chromosome 1, at position 1p36.3. A second Apo-3-positive genomic PAC was mapped to the same position (data not shown). Positive hybridization signals at 1p36.3 were noted at >95% of the cells. Signals were seen in both chromosome 1 homologues in >90% of the positive spreads.

Recent reports disclose that a genomic region which is deleted in certain human neuroblastomas maps within 1p36.2-1p36.3, indicating that a tumor suppressor gene may be present at this locus. Four additional TNFR gene family members, TNFR2, CD30, 4.1 BB and OX40, reside in 1p36 [see Gruss and Dower, supra] but are outside the deleted region [White et al., Proc. Natl. Acad. Sci., 92:5520-5524 (1995)].

Deposit of Material

The following materials have been deposited with the American Type Culture Collection, 12301 Parklawn Drive, Rockville, Md., USA (ATCC):

| Material | ATCC Dep. No. | Deposit Date |
|---|---|---|
| Apo-2LI clone 18.1 | 97493 | Mar. 27, 1996 |
| Apo-3 clone FH20.57 | 55820 | Sept. 5, 1996 |

This deposit was made under the provisions of the Budapest Treaty on the International Recognition of the Deposit of Microorganisms for the Purpose of Patent Procedure and the Regulations thereunder (Budapest Treaty) . This assures maintenance of a viable culture of the deposit for 30 years from the date of deposit. The deposit will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 37 CFR §1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed is:

1. Isolated biologically active Apo-2LI having at least about 80% sequence identity with native sequence Apo-2LI having amino acid residues 1 to 181 of SEQ ID NO:1.

2. The Apo-2LI of claim 1 wherein said Apo-2LI has at least about 90% sequence identity.

3. The Apo-2LI of claim 2 wherein said Apo-2LI has at least about 95% sequence identity.

4. Isolated Apo-2LI comprising amino acid residues 1 to 181 of SEQ ID NO:1.

5. A chimeric molecule comprising the Apo-2LI of claim 1 or claim 4 fused to a heterologous amino acid sequence.

6. The chimeric molecule of claim 5 wherein said heterologous amino acid sequence is an epitope tag sequence.

7. The chimeric molecule of claim 5 wherein said heterologous amino acid sequence is an immunoglobulin sequence.

8. The chimeric molecule of claim 7 wherein said immunoglobulin sequence is an IgG.

9. A dimer molecule comprising a first Apo-2LI and a second Apo-2LI.

10. An antibody which binds to Apo-2LI.

11. The antibody of claim 10 wherein said antibody is a monoclonal antibody.

12. Isolated nucleic acid encoding Apo-2LI.

13. The nucleic acid of claim 12 wherein said nucleic acid encodes an Apo-2LI comprising amino acid residues 1 to 181 of SEQ ID NO:1.

14. A vector comprising the nucleic acid of claim 12.

15. A host cell comprising the vector of claim 14.

16. A method of producing Apo-2LI comprising culturing the host cell of claim 15 and recovering the Apo-2LI from the host cell culture.

17. An article of manufacture, comprising:
a container;
a label on said container; and
a composition contained within said container, said composition comprising Apo-2LI.

18. The article of manufacture of claim 17 further comprising instructions for using the Apo-2LI in vivo or ex vivo.

19. Isolated biologically active Apo-3 polypeptide having at least about 80% sequence identity with native sequence Apo-3 having amino acid residues 1 to 417 of SEQ ID NO:6.

20. The Apo-3 of claim 19 wherein said Apo-3 has at least about 90% sequence identity.

21. The Apo-3 of claim 20 wherein said Apo-3 has at least about 95% sequence identity.

22. Isolated native sequence Apo-3 comprising amino acid residues 1 to 417 of SEQ ID NO:6.

23. Isolated biologically active polypeptide having at least about 80% sequence identity with the extracellular domain sequence of Apo-3 having amino acid residues 1 to 198 of SEQ ID NO:6.

24. The polypeptide of claim 23 wherein said polypeptide has at least about 90% sequence identity.

25. The polypeptide of claim 24 wherein said polypeptide is Apo-2LI.

26. Isolated extracellular domain sequence of Apo-3 comprising amino acid residues 1 to 198 of SEQ ID NO:6.

27. Isolated death domain sequence of Apo-3 comprising amino acid residues 338 to 417 of SEQ ID NO:6.

28. A chimeric molecule comprising the Apo-3 of claim 22 or the extracellular domain sequence of claim 23 fused to a heterologous amino acid sequence.

29. The chimeric molecule of claim 28 wherein said heterologous amino acid sequence is an epitope tag sequence.

30. The chimeric molecule of claim 28 wherein said heterologous amino acid sequence is an immunoglobulin sequence.

31. The chimeric molecule of claim 30 wherein said immunoglobulin sequence is an IgG.

32. An antibody which binds to Apo-3 or to the extracellular domain sequence of claim 23.

33. The antibody of claim 32 wherein said antibody is a monoclonal antibody.

34. Isolated nucleic acid encoding the Apo-3 of claim 22, the extracellular domain sequence of claim 23 or the death domain sequence of claim 27.

35. The nucleic acid of claim 34 wherein said nucleic acid encodes native sequence Apo-3 comprising amino acid residues 1 to 417 of SEQ ID NO:6.

36. A vector comprising the nucleic acid of claim 34.

37. The vector of claim 36 operably linked to control sequences recognized by a host cell transformed with the vector.

38. A host cell comprising the vector of claim 36.

39. A process of using a nucleic acid molecule encoding Apo-3 to effect production of Apo-3 comprising culturing the host cell of claim 38.

40. A non-human, transgenic animal which contains cells that express nucleic acid encoding Apo-3.

41. The animal of claim 40 which is a mouse or rat.

42. A non-human, knockout animal which contains cells having an altered gene encoding Apo-3.

43. The animal of claim 42 which is a mouse or rat.

44. An article of manufacture, comprising a container and a composition contained within said container, wherein the composition includes Apo-3 polypeptide or Apo-3 antibodies.

45. The article of manufacture of claim 44 further comprising instructions for using the Apo-3 polypeptide or Apo-3 antibodies in vivo or ex vivo.


SEQUENCE LISTING (11 sequences)

SEQ ID NO: 1 - protein, 181 aa
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPT

SEQ ID NO: 2 - DNA, 433 bp; Single-stranded, Linear
ctgctgggggcccgggccagnggcggcactcgtagccccaggtgtgactgtgccggtgacttccacaagaagattggtctgttttgttgcagaggctgcccagcggggcaactacctgaaggccccttgcacggagccctgcgcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagcagggctggtttgtggagtgccagggtcagccaatgtgtcagcagtttcacccttctaatgccaaccatgcctagactgcggggccctgcaacgcaacacacggctaatntgtttcccgcagagatnattgtt

SEQ ID NO: 3 - DNA, 28 bp; Single-stranded, Linear
cccgctgccaggcctgtgatgagcaggc

SEQ ID NO: 4 - DNA, 28 bp; Single-stranded, Linear
cagggccccgcagtctaggcatggttgg

SEQ ID NO: 5 - DNA, 1438 bp; Single-stranded, Linear
gaattccggcgcggaggccgagagagaagtcacttgccctggctctaccttgaagtggttctcagggttggggcgagagtcggggtggggaccgagatgcagctctatcctgtgcccctggtcgcagcaggcagcccagcgcttcgcgtgttctacttggcctgtccgctgccgcctaatgagctcaggtctaggccgagcagagggggcacctggtcggactcggttgggctcgggcggccccgcctccccccgcccgccaggcgggcccttctcgacggcgcggggcgggccctgcgggcgcggggctgaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgtaattcctagctgtcgtgggatggagggaagggcggctgggagcagagcaggggcctggggtggggcaggtgctgctggttcaggaataggaagaggggatagggaggagggagccttggccctgtgatgggtgggccccacttcaggcaaacttagatggcaaaagagcaatctggatccgccttagccagatacataagggtatttgccttcactttcagccagcattccccccagcgatcctagccagatattacagatgatttgtcacttacacagagagtcacattgatatagctttaaaacttgggctgaaggaggttgaggctgcagtgagctatgatcgtgccactgcacttcagcctgggcaacagagcgagacctattaaataaataaataaatattaaatctattaaatattaaatattaaatctattaaataaataaatacaaagggctgagagtcaggactgtgctgctagttctctaggggatcttgggcaagtgcagagaattc

SEQ ID NO: 6 - protein, 417 aa
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 7 - DNA, 27 bp; Single-stranded, Linear
ggcgctctggtggcccttgcagaagcc

SEQ ID NO: 8 - DNA, 25 bp; Single-stranded, Linear
ttcggccgagaagttgagaaatgtc

SEQ ID NO: 9 - DNA, 1634 bp; Single-stranded, Linear
cgggccctgcgggcgcggggctgaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtgacacggcgcccacttgccacctaggcgctctggtggcccttgcagaagccctaagtacggttacttatgcgtgtagacattttatgtcacttattaagccgctggcacggccctgcgtagcagcaccagccggccccacccctgctcgcccctatcgctccagccaaggcgaagaagcacgaacgaatgtcgagagggggtgaagacatttctcaacttctcggccggagtttggctgagatcgcggtattaaatctgtgaaagaaaacaaaaaaaaaaaaaaaaaaaaaaaa

SEQ ID NO: 10 - DNA, 29 bp; Single-stranded, Linear
atcagggactttccgctggggactttccg

SEQ ID NO: 11 - DNA, 30 bp; Single-stranded, Linear
aggatgggaagtgtgtgatatatccttgat